SEQUENCE LISTING (2 sequences)

SEQ ID NO: 1 - DNA, 21 bp, Artificial Sequence
  Description of Artificial Sequence Synthetic oligonucleotide
cuuacgcugaguacuucgatt

SEQ ID NO: 2 - DNA, 21 bp, Artificial Sequence
  Description of Artificial Sequence Synthetic oligonucleotide
ucgaaguacucagcguaagtt

(19) United States
(12) Patent Application Publication
(10) Pub. No.: US 2015/0174549 A1
(43) Pub. Date: Jun. 25, 2015

(54) HIGH-THROUGHPUT SYNTHESIS OF NANOPARTICLES

(71) Applicants: Jong-Min Lim, Somerville, MA (US); Laura Marie Gilson, Moraga, CA (US); Sunandini Chopra, Cambridge, MA (US); Omid Cameron Farokhzad, Waban, MA (US); Rohit Nandkumar Karnik, Cambridge, MA (US); Archana Swami, Everett, MA (US)

(72) Inventors: Jong-Min Lim, Somerville, MA (US); Laura Marie Gilson, Moraga, CA (US); Sunandini Chopra, Cambridge, MA (US); Omid Cameron Farokhzad, Waban, MA (US); Rohit Nandkumar Karnik, Cambridge, MA (US); Archana Swami, Everett, MA (US)

(73) Assignees: THE BRIGHAM AND WOMEN'S HOSPITAL CORPORATION, BOSTON, MA (US); MASSACHUSETTS INSTITUTE OF TECHNOLOGY, Cambridge, MA (US)

(21) Appl. No.: 14/523,869

(22) Filed: Oct. 25, 2014

Related U.S. Application Data
(60) Provisional application No. 61/895,594, filed on Oct. 25, 2013.

(51) Int. Cl. B01J 19/06; A61K 9/51; B01F 15/02; A61K 31/713; A61K 47/48; B01J 19/26; C01G 49/02; C09K 11/02

(52) U.S. Cl. CPC B01J 19/06; C01G 49/02; A61K 9/5192; A61K 9/5153; C09K 11/02; A61K 31/713; A61K 47/48853; A61K 47/48192; B01J 19/26; B01F 15/0254; B01J 2219/247; B01F 2215/0036; B01F 2215/0032

(57) ABSTRACT

A simple and versatile coaxial turbulent jet mixer can synthesize a range of nanoparticles at high throughput, while maintaining the advantages of homogeneity, reproducibility, and tunability that are normally accessible only in specialized microscale mixing devices. Rapid mixing down to a timescale of 7 ms can be achieved by controlling the Reynolds number, providing homogeneous and controllable environments for formation of nanoparticles, for example, by precipitation. The device fabrication does not require specialized machining, making it accessible for a wide range of biomedical laboratories.

### CLAIM OF PRIORITY

This application claims the benefit of prior U.S. Provisional Application No. 61/895,594 filed on Oct. 25, 2013, which is incorporated by reference in its entirety.

### GOVERNMENT SPONSORSHIP

This invention was made with government support under Grant Nos. EB015419 and CA151884 awarded by the National Institutes of Health. The government has certain rights in the invention.

### TECHNICAL FIELD

The present invention relates to a micromixers and nanoparticles.

### SEQUENCE LISTING

The instant application contains a Sequence Listing which has been submitted electronically in ASCII format and is hereby incorporated by reference in its entirety. Said ASCII copy, created on Feb. 27, 2015, is named 14952.0462 SL.txt and is 1,053 bytes in size.

### BACKGROUND

Nanoparticles are promising for various applications including biomedical, energy, catalysis, cosmetics, foods, displays, and semiconductor industry. The physicochemical properties of nanoparticles (e.g., composition, size, shape, size distribution, and surface functional group) can be controllable to meet the various needs in the wide range of applications. Recently, microfluidic platform can enhance the controllability and reproducibility of synthesized nanoparticles compared to the conventional bulk synthesis method, because the microfluidic platform can offer precisely controlled reaction environments. However, the productivity of microfluidic systems is lower than that of batch reactors due to low flow rates, which can limit the application of nanoparticles synthesized by microfluidic systems.

### SUMMARY

A method for preparing nanoparticles can include flowing a first stream of a first solution into a conduit, wherein the first solution contains precursors of the nanoparticles, flowing a second stream of a second solution into the conduit, and mixing the first stream and the second stream to form a mixed stream having a Reynolds number of between 300 and 1,000,000 in which the nanoparticles are formed. In certain embodiments, the conduit can be a tube. In certain other embodiments, the formation of the nanoparticles can be continuous. In certain circumstances, the second stream can flow simultaneously with the first stream. In certain other circumstances, the first stream is introduced within the second stream. In certain circumstances, the second stream can contain precursors of nanoparticles.

In certain embodiments, the nanoparticles can be substantially uniformly distributed in the mixed stream after formation. The mixed stream can include a vortex regime, a turbulence regime, or a turbulent jet regime. The flow behavior of the mixed stream can include turbulent jet flow. The flow velocity and the Reynolds number of the mixed stream can vary. A mixing timescale of the mixed stream can be between 0.1 and 100 milliseconds. A flow velocity ratio of the first stream to the second stream can be between 0.01 and 100. A volume ratio between the first solution and the second solution can be between 10:1 and 1:100. The method of claim 1, wherein the volume ratio between the first solution and the second solution is between 1:3 to 1:20.

In certain other embodiments, the nanoparticles can be formed by nanoprecipitation. In certain other embodiments, a component of the first solution can react with a component of the second solution.

In certain other embodiments, the cross sectional area of the first stream is more than 1%, more than 10%, more than 20%, more than 30%, more than 40%, more than 50%, more than 60%, more than 70%, or more than 80% of the cross sectional area of the conduit. The cross sectional area of the first stream can be less than 90%, less than 80%, less than 70%, less than 60%, less than 50%, less than 40%, less than 30%, less than 20%, or less than 10% of the cross sectional area of the conduit.

In certain other embodiments, the size of the nanoparticles can be between 1 nm and 500 nm. The size of the nanoparticles can be changed by changing the flow parameters of the first stream and/or the second stream. In other circumstances, the composition, shape, size distribution, or surface functional group can be changed by changing the flow parameters of the first stream and/or the second stream.

In certain other embodiments, the nanoparticles can include PLGA-PEG, iron oxide, polystyrene, siRNA/PEI polyplex, or lipid vesicles. In certain other embodiments, the nanoparticles can contain a drug molecule, or a fluorescent molecule.

In certain circumstances, a device for preparing nanoparticle can include a conduit configured to introduce a first stream of a first solution into the conduit, a second stream of a second solution into the conduit at a mixing zone of the conduit, wherein the Reynolds number at the mixing zone is between 300 and 1,000,000. In certain embodiments, the device can be a coaxial turbulent jet mixer.

In certain other circumstances, a device for preparing nanoparticles can include a first conduit configured to introduce a first stream of a first solution into the first conduit, and a second conduit configured to introduce a second stream of a second solution into the second conduit, wherein the first conduit is inserted into the second conduit, and wherein the Reynolds number at the location of the introduction of the first solution is between 300 and 1,000,000. The device can further include a third conduit configured to introduce a third stream of a third solution, wherein the third conduit is inserted into the second conduit, and wherein the Reynolds number at the location of the introduction of the third solution is between 300 and 1,000,000.

In certain embodiments, the location of the introduction of the first solution, the location of the introduction of the second solution, and the location of the introduction of third solution are controlled to control the time delay between the introduction of the first solution, the introduction of the second solution, and the introduction of the third solution.

In certain other embodiments, the device can include a plurality of devices, each device comprising a first conduit configured to introduce a first stream of a first solution into the first conduit, and a second conduit configured to introduce a second stream of a second solution into the second conduit, wherein the first conduit is inserted into the second conduit, and wherein the Reynolds number at the location of the introduction of the first solution is between 300 and 1,000,000.

In another aspect, a method for preparing nanoparticles can include introducing a first stream of a first solution into a first conduit, wherein the Reynolds number at the location of the introduction of the first solution is between 300 and 1,000,000, introducing a second stream of a second solution into a second conduit, wherein the first conduit is inserted into the second conduit, introducing a third stream of a third solution into a third conduit, wherein the third conduit is inserted into the second conduit, and wherein the Reynolds number at the location of the introduction of the third solution is between 300 and 1,000,000, wherein the first solution and/or the second solution contains nanoparticle precursors, and wherein nanoparticles form when the first solution mixes with the second solution and the third solution.

In another aspect, a method for preparing nanoparticles can include continuously flowing a first stream of a first solution into a conduit, wherein the first solution contains precursors of the nanoparticles, and continuously flowing a second stream of a second solution into the conduit such that the second stream forms a turbulent jet within the first stream, wherein the first stream and the second stream form a mixed stream having a Reynolds number of between 300 and 1,000,000 in which the nanoparticles are formed.

Other aspects, embodiments, and features will be apparent from the following description, the drawings, and the claims.

### BRIEF DESCRIPTION OF THE DRAWINGS

FIG. 1A is a schematic illustration of the coaxial turbulent jet mixer for high-throughput synthesis of nanoparticles with turbulence induced by vortex. FIG. 1B is a schematic illustration of the coaxial turbulent jet mixer for high-throughput synthesis of nanoparticles with turbulence induced by jetting. The insets show the top view of the turbulent jets with different flow regimes.

FIG. 2A is a photograph depicting coaxial turbulent jet mixer made from various sizes of syringe needles and clear polycarbonate tee union tube fittings. FIG. 2B is a photograph depicting a coaxial turbulent jet mixer made from 23G needle and ⅛″ PTFE tee union tube fittings. FIG. 2C is a schematic drawing of geometry and flow condition of the coaxial turbulent jet mixer.

FIG. 3 is a phase diagram of jet flow regime in terms of R and Re.

FIG. 4A is a graph depicting mixing time as a function of Re when the coaxial turbulent jet mixer is operated in turbulent jet regime. Inset shows the method for determining L. FIG. 4B is a graph depicting non-dimensionalized mixing time as a function of Re when the coaxial turbulent jet mixer is operated in turbulent jet regime.

FIG. 5A is a TEM image depicting PLGA-PEG nanoparticles prepared using the coaxial turbulent jet mixer. FIG. 5B is a graph depicting the size distribution of the PLGA-PEG nanoparticles by volume fraction. FIG. 5C is a graph depicting the effect of Re on the size of the PLGA-PEG nanoparticles.

FIG. 6A is a TEM image depicting lipid vesicles prepared using the coaxial turbulent jet mixer. FIG. 6B is a graph depicting the size distribution of the lipid vesicles by volume fraction. FIG. 6C is a graph depicting the effect of Re on the size of the lipid vesicles.

FIG. 7A is a TEM image depicting iron oxide nanoparticles prepared using the coaxial turbulent jet mixer. FIG. 7B is a graph depicting the size distribution of iron oxide nanoparticles by volume fraction. FIG. 7C is a graph depicting the effect of Re on the size of iron oxide nanoparticles.

FIG. 8A is a TEM image depicting the polystyrene nanoparticles prepared using the coaxial turbulent jet mixer. FIG. 8B is a graph depicting the size distribution of the polystyrene nanoparticles by volume fraction. FIG. 8C is a graph depicting the effect of Re on the size of the polystyrene nanoparticles.

FIG. 9A is a graph depicting the size distribution of the docetaxel loaded PLGA-PEG nanoparticles prepared using the coaxial turbulent jet mixer and bulk synthesis method. FIG. 9B is a graph depicting the drug loading and encapsulation efficiency of docetaxel loaded PLGA-PEG nanoparticles obtained by coaxial turbulent jet mixer.

FIG. 10A is a graph depicting the size distribution of the insulin loaded PLGA-PEG nanoparticles prepared using the coaxial turbulent jet mixer. FIG. 10B is a graph depicting the drug loading and encapsulation efficiency of insulin loaded PLGA-PEG nanoparticles obtained by coaxial turbulent jet mixer.

FIG. 11A is a graph depicting the size distribution of the fluorescent lipid vesicles prepared using the coaxial turbulent jet mixer. FIG. 11B is a photograph and fluorescence microscope image of lipid vesicles with DiIC₁₈ dye.

FIG. 12A is a graph depicting the size distribution of the fluorescent polystyrene nanoparticles prepared using the coaxial turbulent jet mixer. FIG. 12B is a photograph and fluorescence microscope image of polystyrene nanoparticles with perylene dye.

FIG. 13A is a graph depicting the size distribution of the siRNA/PEI polyplex nanoparticles prepared using the coaxial turbulent jet mixer and bulk synthesis method. Inset is a TEM image of siRNA/PEI polyplex nanoparticles. FIG. 13B is a graph depicting the luciferase expression (%) in HeLa cells expressing both firefly and renilla luciferase, treated with nanoparticles carrying GL3 siRNA, at various effective siRNA concentrations relative to scrambled siRNA, used as control.

FIG. 14 is a schematic drawing of an embodiment of a mixer.

FIG. 15 is a schematic drawing of an embodiment of a mixer.

FIG. 16 is a schematic drawing of an embodiment of a mixer.

FIG. 17 is a schematic drawing of an embodiment of a mixer.

FIG. 18 is a graph depicting the size distribution by volume fraction of polystyrene nanoparticles prepared using the coaxial turbulent jet mixer and commercially available nanoparticles.

FIG. 19 is schematic illustrations and top views of fluid flow at laminar (R=1 and Re=237), vortex and turbulence (R=0.3 and Re=353), and turbulent jet (R=10 and Re=1311) regimes.

FIGS. 20A-20F depicts characteristics of iron oxide nanoparticles prepared using coaxial turbulent jet mixer and bulk synthesis method. FIGS. 20A and 20D are TEM images, FIGS. 19B and 20E are graphs depicting size distribution by number fraction obtained from TEM image, and FIGS. 20C and 20F are graphs depicting size distribution by volume fraction from TEM image and dynamic light scattering of iron oxide nanoparticles prepared by coaxial turbulent jet mixer (FIGS. 20A-20C) and bulk synthesis method (FIGS. 20D-20F), respectively.

FIGS. 21A-21F depicts charateristics of PLGA₉₅ₖ-PEG₅ₖ NPs prepared using coaxial turbulent jet mixer in tens of milligram scale and in a few gram scale. FIGS. 21A and 21D are TEM image, FIGS. 21B and 21E are graphs depicting size distribution by number fraction obtained from TEM image, and FIGS. 21C and 21F are graphs depicting size distribution by volume fraction from TEM image and dynamic light scattering of PLGA₉₅ₖ-PEG₅ₖ NPs prepared using coaxial turbulent jet mixer (FIGS. 21A-21C) in tens of milligram scale and (FIGS. 21D-21F) in a few gram scale, respectively.

### DETAILED DESCRIPTION

Nanoparticles (NPs) have shown great promise for various biomedical applications including nanocarriers for drug delivery, fluorescence imaging, and magnetic resonance imaging (MRI) contrast agents. See, Valencia, P. M., Farokhzad, O. C., Karnik, R. & Langer, R. Microfluidic technologies for accelerating the clinical translation of nanoparticles. Nat. Nanotechnol. 7, 623-629 (2012), Kamaly, N., Xiao, Z., Valencia, P. M., Radovic-Moreno, A. F. & Farokhzad, O. C. Targeted polymeric therapeutic nanoparticles: design, development and clinical translation. Chem. Soc. Rev. 41, 2971-3010 (2012), Santra, S., Dutta, D., Walter, G. A. & Moudgil, B. M. Fluorescent nanoparticle probes for cancer imaging. Technol. Cancer Res. T. 4, 593 (2005), Rao, J., Dragulescu-Andrasi, A. & Yao, H. Fluorescence imaging in vivo: recent advances. Curr. Opin. Biotech. 18, 17-25 (2007), Santra, S. & Malhotra, A. Fluorescent nanoparticle probes for imaging of cancer. Wiley Interdisciplinary Reviews: Nanomedicine and Nanobiotechnology 3, 501-510, doi:10.1002/wnan.134 (2011), and Qiao, R., Yang, C. & Gao, M. Superparamagnetic iron oxide nanoparticles: from preparations to in vivo MRI applications. J. Mater. Chem. 19, 6274-6293 (2009), each of which is incorporated by reference in its entirety. Indeed, liposome (DOXIL) and protein based drug delivery system (Abraxane) for cancer therapy and iron oxide nanoparticles (Ferumoxide) for MRI contrast agent were approved by FDA. In addition, polymeric micelle nanoparticle (Genexol-PM) was approved in Korea and in phase II clinical development in the USA. See, Shi, J., Xiao, Z., Kamaly, N. & Farokhzad, O. C. Self-Assembled Targeted Nanoparticles: Evolution of Technologies and Bench to Bedside Translation. Accounts of Chemical Research 44, 1123-1134, doi:10.1021/ar200054n (2011), which is incoporated by reference in its entirety.

Despite these advances, translation of NPs from the bench to bedside is difficult; among the major challenges is controlling the properties and quality of NPs from laboratory scale synthesis to the clinical production scale. See, Valencia, P. M.; Farokhzad, 0. C.; Karnik, R.; Langer, R. Microfluidic Technologies for Accelerating the Clinical Translation of Nanoparticles. Nat. Nanotechnol. 2012, 7, 623-629, which is incorporated by reference in its entirety. While nanoparticles are conventionally synthesized by batch type reactors, these bulk synthesis methods tend to have limited batch-to-batch reproducibility and controllability in terms of physicochemical properties of the synthesized nanoparticles. In addition, the scaling up of batch procedures for industrial-scale production needs considerable trial and error for process optimization. See, Song, Y. J., Hormes, J. & Kumar, C. Microfluidic synthesis of nanomaterials. Small 4, 698-711 (2008), and Marre, S. & Jensen, K. F. Synthesis of micro and nanostructures in microfluidic systems. Chem. Soc. Rev. 39, 1183-1202 (2010), each of which is incorporated by reference in its entirety. Because the in vivo fate of nanoparticles is strongly dependent on their physicochemical properties, the development of novel methods that can synthesize nanoparticles in a reproducible and controlled manner from laboratory-scale in vivo studies all the way to production scale is critical for the translation of nanoparticles to the clinic. See, Hrkach, J. et al. Preclinical development and clinical translation of a PSMA-targeted docetaxel nanoparticle with a differentiated pharmacological profile. Sci. Transl. Med. 4, 128ra139 (2012), which is incorporated by reference in its entirety.

Continuous synthesis of nanoparticles tends to have better reproducibility and controllability compared to batch-type bulk synthesis methods. See, Wagner, J. & Köhler, J. M. Continuous Synthesis of Gold Nanoparticles in a Microreactor. Nano Lett. 5, 685-691, doi:10.1021/n1050097t (2005), Jahn, A. et al. Preparation of nanoparticles by continuous-flow microfluidics. J. Nanopart. Res. 10, 925-934 (2008), and Krishnadasan, S., Yashina, A., deMello, A. J. & deMello, J. C. in Advances in Chemical Engineering Vol. Volume 38 (ed J. C. Schouten) 195-231 (Academic Press, 2010), each of which is incorporated by reference in its entirety. Recently, microfluidic platforms have been developed to enhance the controllability and reproducibility of synthesized nanoparticles because of the ability of microfluidics to offer precisely controlled reaction environments. See, Yeo, L. Y., Chang, H. C., Chan, P. P. Y. & Friend, J. R. Microfluidic devices for bioapplications. Small 7, 12-48 (2011), which is incorporated by reference in its entirety. Recently, controlled nanoparticle synthesis by rapid nanoprecipitation have been demonstrated using various poly(dimethylsiloxane) (PDMS) microfluidic devices including 2D hydrodynamic flow focusing (HFF), 3D HFF, herringbone micromixer, and mixing by microvortices. See, Chen, D. et al. Rapid discovery of potent siRNA-containing lipid nanoparticles enabled by controlled microfluidic formulation. J. Am. Chem. Soc. 134, 6948-6951 (2012), Jahn, A., Vreeland, W. N., Gaitan, M. & Locascio, L. E. Controlled vesicle self-assembly in microfluidic channels with hydrodynamic focusing. J. Am. Chem. Soc. 126, 2674-2675 (2004), Karnik, R. et al. Microfluidic platform for controlled synthesis of polymeric nanoparticles. Nano Lett. 8, 2906-2912 (2008), Karnik, R. et al. Microfluidic Synthesis of Organic Nanoparticles. US 2010/0022680 (2010), Jahn, A. et al. Microfluidic mixing and the formation of nanoscale lipid vesicles. ACS Nano 4, 2077-2087 (2010), Rhee, M. et al. Synthesis of size-tunable polymeric nanoparticles enabled by 3D hydrodynamic flow focusing in single-layer microchannels. Adv. Mater. 23, H79-H83 (2011), Zhigaltsev, I. V. et al. Bottom-up design and synthesis of limit size lipid nanoparticle systems with aqueous and triglyceride cores using millisecond microfluidic mixing. Langmuir 28, 3633-3640 (2012), and Kim, Y. T. et al. Mass production and size control of lipid-polymer hybrid nanoparticles through controlled microvortices. Nano Lett. 12, 3587-3591 (2012), each of which is incorporated by reference in its entirety.

However, there are several intrinsic limitations on microfluidic systems for the synthesis of nanoparticles. The requirement for specialized microfabrication facilities, lack of robustness, and know-how required to operate the devices creates a barrier for their utilization in typical biomedical research laboratories. See, Lohse, S. E., Eller, J. R., Sivapalan, S. T., Plews, M. R. & Murphy, C. J. A simple millifluidic benchtop reactor system for the high-throughput synthesis and functionalization of gold nanoparticles with different sizes and shapes. ACS Nano 7, 4135-4150 (2013), which is incorporated by reference in its entirety. Second, only a handful of organic solvents are compatible with conventional PDMS microfluidic systems, which hinders PDMS microfluidic systems from serving as versatile platforms for synthesis of various types of nanoparticles. See, Lee, J. N., Park, C. & Whitesides, G. M. Solvent compatibility of poly(dimethylsiloxane)-based microfluidic devices. Anal. Chem. 75, 6544-6554 (2003), which is incorporated by reference in its entirety. Third, productivity of microfluidic systems is typically low (<0.3 g/h), which considerably falls short of the production rates typically required in clinical studies and industrial scale production. While millifluidic systems including confined impinging jets mixer, multi-inlet vortex mixer, and Y-mixer have been used for the synthesis of nanoparticles, complicated micromachining is still required. See, Kim, Y. T. et al. Mass production and size control of lipid-polymer hybrid nanoparticles through controlled microvortices. Nano Lett. 12, 3587-3591 (2012), Johnson, B. K. & Prud'homme, R. K. Mechanism for rapid self-assembly of block copolymer nanoparticles. Phys. Rev. Lett. 91, 118302 (2003), Zhang, C., Pansare, V. J., Prud'homme, R. K. & Priestley, R. D. Flash nanoprecipitation of polystyrene nanoparticles. Soft Matter 8, 86-93 (2012), and Shen, H., Hong, S., Prud'homme, R. & Liu, Y. Self-assembling process of flash nanoprecipitation in a multi-inlet vortex mixer to produce drug-loaded polymeric nanoparticles. J. Nanopart. Res. 13, 4109-4120 (2011), each of which is incorporated by reference in its entirety. This makes it difficult for biomedical research laboratories to use these devices for development of nanoparticles, and consequently there are only a few studies using nanoparticles synthesized using these devices.

Recently, nanoparticle synthesis using millifluidic systems have been demonstrated to overcome some of the drawbacks in microfluidic nanoparticle synthesis systems. Recently, Prud'homme's group developed various millifluidic apparatus for preparation of polymeric nanoparticles using flash nanoprecipitation. See, Johnson, B. K. & Prud'homme, R. K. Mechanism for rapid self-assembly of block copolymer nanoparticles. Phys. Rev. Lett. 91, 118302 (2003), Johnson, B. K. & Prud'homme, R. K. Process and apparatuses for preparing nanoparticle compositions with amphiphilic copolymers and their use. US 2004/0091546 (2004), Shen, H., Hong, S., Prud'homme, R. & Liu, Y. Self-assembling process of flash nanoprecipitation in a multi-inlet vortex mixer to produce drug-loaded polymeric nanoparticles. J. Nanopart. Res. 13, 4109-4120 (2011), and Zhang, C., Pansare, V. J., Prud'homme, R. K. & Priestley, R. D. Flash nanoprecipitation of polystyrene nanoparticles. Soft Matter 8, 86-93 (2012), each of which is incorporated by reference in its entirety. Because the millifluidic mixing apparatus could be made of materials with resistant to various organic solvents, various polymeric nanoparticles including amphiphilic block copolymer nanoparticles and polystyrene nanoparticles could be prepared using the millifluidic mixing apparatus. However, complicated micromachining is still required for the fabrication of millifluidic mixing apparatus. Precipitating precursors easily come into contact with the device wall, which may cause aggregations under some operating conditions. Flow visualization is typically hard to achieve due to opacity and complex geometry. In addition, the production rates are not quantified in their reports. Abou-Hassan et al. also developed millifluidic coaxial flow device by fixing a glass capillary in PDMS channel. See, Abou Hassan, A., Sandre, O., Cabuil, V. & Tabeling, P. Synthesis of iron oxide nanoparticles in a microfluidic device: preliminary results in a coaxial flow millichannel. Chem. Commun. 1783-1785 (2008), and Abou-Hassan, A. et al. Fluorescence confocal laser scanning microscopy for pH mapping in a coaxial flow microreactor: application in the synthesis of superparamagnetic nanoparticles. J. Phys. Chem. C 113, 18097-18105 (2009), each of which is incorporated by reference in its entirety. However, because of the aforementioned intrinsic limitations of PDMS channel, their coaxial flow device could not serve as a versatile platform for nanoparticle synthesis. In addition, their coaxial flow device had limited production rates because the device was operated only in laminar flow regime. Coaxial flow device, which was operated in turbulent flow regime, was reported by Baldyga group. See, Baldyga, J. & Henczka, M. Turbulent mixing and parallel chemical reactions in a pipe. Recents Progres en Genie des Procedes 11, 341-348 (1997), Henczka, M. Influence of turbulent mixing on the course of homogeneous chemical reacions—closure hypothesis. Ph.D. Thesis, Warsaw University of Technology (1997), and Baldyga, J. & Bourne, J. R. Turbulent mixing and chemical reactions. (John Wiley & Sons, 1999), each of which is incorporated by reference in its entirety. However, they only focused on the characterization of turbulent mixing in coaxial pipe. Thus they did not apply the technology for the synthesis of nanoparticles. More recently, Lohse et al. developed simple millifluidic reactor assembled by commercially available components for controlled synthesis of gold nanoparticles. See, Lohse, S. E., Eller, J. R., Sivapalan, S. T., Plews, M. R. & Murphy, C. J. A simple millifluidic benchtop reactor system for the high-throughput synthesis and functionalization of gold nanoparticles with different sizes and shapes. ACS Nano 7, 4135-4150 (2013), which is incorporated by reference in its entirety. However, the production rates of their simple millifluidic reactor are still comparable with high-throughput microfluidic nanoparticle synthesis system (˜0.005 g/min). See, Kim, Y. T. et al. Mass production and size control of lipid-polymer hybrid nanoparticles through controlled microvortices. Nano Lett. 12, 3587-3591 (2012), which is incorporated by reference in its entirety. Considering that the production rates typically required in clinical studies and industrial scale of nanoparticles are order of 0.1 kg/day and 1 kg/day, current nanoparticle synthesis platforms other than batch type conventional bulk synthesis methods cannot meet the requirements.

A coaxial turbulent jet mixer can be used continuously produce nanoparticles. The mixer can be used to prepare nanoparticles by introducing a first flow stream into a second flow stream. When the two streams are arranged to have coaxial flow at differential rates, turbulent conditions are created that induce rapid mixing. Either one of the first flow steam and the second flow stream or both the first and the second streams can contain precursors of nanoparticles. When the compositions of the streams include materials that will form particles by precipitation, crystallization or reaction between the components in a controlled manner such that particle growth stops at nanometer particle sizes, a high volume of narrow size distribution particles can be produced. The streams flow through a conduit, which directed the fluid from the precursor source to an output location.

The mixing zone can have a high Reynolds number. For example, the Reynolds number can be between 300 and 1,000,000. The flow velocity ratio can be greater than 1.

Nanoparticle precursors can have compositions that can be used to form or assist in formation of nanoparticles. For example, a stream with different pH or salt compositions can trigger precipitation. In certain embodiments, a component of one solution interacts with component of the other solution can trigger formation of nanoparticles. For example, mixing of calcium chloride with sodium alginate can trigger precipitation.

The formation of the nanoparticles can be continuous. For example, the conduit can be a tube or other channel through which the fluids can flow. The properties of the nanoparticles can be controlled by adjusting flow parameters of the streams. For example, size, composition, shape, size distribution, or surface functional group of the nanoparticles can be changed by changing the flow parameters of the first stream and/or the second stream.

The mixed stream can form a vortex regime, a turbulence regime or a turbulent jet regime, which can be accomplished by varying the flow velocity of the streams, the Reynolds number, or the relative cross sectional areas of the flow streams. For example, the cross sectional area of the first stream can be more than 1% of the cross sectional area of the conduit. In another example, the cross sectional area of the first stream can be less than 90% of the cross sectional area of the conduit. The cross sectional area ratio can be 10:1 to 1:10, 1:5 to 5:1, 1:3 to 3:1, 1:2 to 2:1 or 1:1. The geometry of the tip of the inner tube may be adjusted to affect the flow. For example, an inner tube with a thicker wall and sharp corners is expected to trigger turbulence more easily.

In certain other circumstances, a device for preparing nanoparticles can include a first conduit configured to introduce a first stream of a first solution into the first conduit, and a second conduit configured to introduce a second stream of a second solution into the second conduit, wherein the first conduit is inserted into the second conduit, and wherein the Reynolds number at the location of the introduction of the first solution is between 300 and 1,000,000. The device can further include a third conduit configured to introduce a third stream of a third solution, wherein the third conduit is inserted into the second conduit, and wherein the Reynolds number at the location of the introduction of the third solution is between 300 and 1,000,000. In certain embodiments, the third conduit can be inserted coaxially into the first conduit.

Disclosed herein is a simple and versatile coaxial turbulent jet mixer for synthesizing nanoparticles with high production rates up to 3.15 kg/day and 1.15 ton/yr suitable for in vivo studies, clinical trials, and industrial scale production, while retaining the advantages of better homogeneity and control over nanoparticle properties due to rapid mixing that are normally accessible only by using specialized micro-fabricated devices. The mixer consists of coaxial cylindrical tubes where nanoparticle precursors and solvent/anti-solvent are injected. The high Reynolds number (Re) (>500) results in turbulent flow that rapidly mixes the injected solutions via the formation of a turbulent jet. Because of the rapid solvent exchange, uniform nanoparticles could be synthesized by self-assembly of the raw materials. The coaxial turbulent jet mixer can be prepared in half an hour with off-the-shelf components and a drill. The versatility of the coaxial turbulent jet mixer is demonstrated by preparing various types of nanoparticles including poly(lactide-co-glycolide)-b-polyethyleneglycol (PLGA-PEG) nanoparticles, lipid vesicles, iron oxide nanoparticles, polystyrene nanoparticles, and siRNA-polyelectrolyte (Polyethyleneimine: PEI_{25K, Branched}) polyplex nanoparticles by rapid nanoprecipitation encapsulating different functional agents including anticancer drug, insulin, fluorescent dyes, and siRNA. See, Karnik, R. et al. Microfluidic platform for controlled synthesis of polymeric nanoparticles. Nano Lett. 8, 2906-2912 (2008), Rhee, M. et al. Synthesis of size-tunable polymeric nanoparticles enabled by 3D hydrodynamic flow focusing in single-layer microchannels. Adv. Mater. 23, H79-H83 (2011), Jahn, A., Vreeland, W. N., Gaitan, M. & Locascio, L. E. Controlled vesicle self-assembly in microfluidic channels with hydrodynamic focusing. J. Am. Chem. Soc. 126, 2674-2675 (2004), Jahn, A. et al. Microfluidic mixing and the formation of nanoscale lipid vesicles. ACS Nano 4, 2077-2087 (2010), Abou Hassan, A., Sandre, O., Cabuil, V. & Tabeling, P. Synthesis of iron oxide nanoparticles in a microfluidic device: preliminary results in a coaxial flow millichannel. Chem. Commun. 0, 1783-1785 (2008), and Abou-Hassan, A. et al. Fluorescence confocal laser scanning microscopy for pH mapping in a coaxial flow microreactor: application in the synthesis of superparamagnetic nanoparticles. J. Phys. Chem. C 113, 18097-18105 (2009), each of which is incorporated by reference in its entirety.

### Fabrication of Coaxial Turbulent Jet Mixer

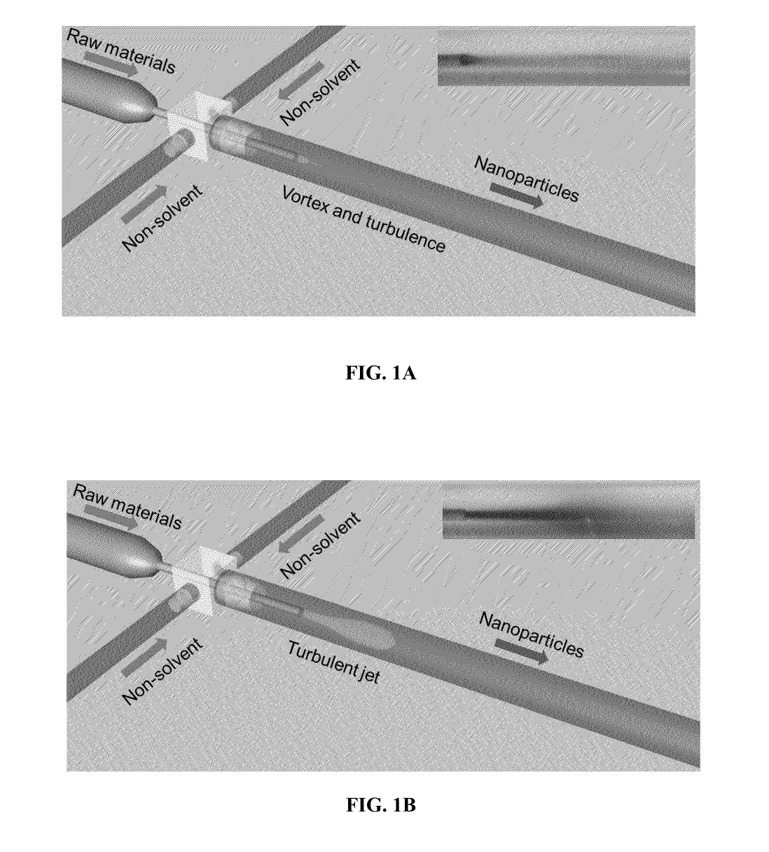

The mixer consists of coaxial cylindrical tubes where nanoparticle precursors and non-solvent are injected through the inner and outer tubes, respectively. In certain circumstances, the second stream can also contain precursors of nanoparticles. The high Reynolds number (Re) results in turbulent flow that rapidly mixes the injected solutions by the formation of a turbulent jet (FIG. 1). Because of the rapid solvent exchange, uniform nanoparticles can be synthesized by self-assembly of the raw materials in a process known as nanoprecipitation.

The tee union tube fittings made of clear polycarbonate (McMaster-Carr) or PTFE (Plasmatech Co.) were used for fabrication. A hole was drilled using a 0.025 inch diameter drill bit (#72, Drill bit city) and a 23 G blunt needle (337 μm I.D. and 641.4 μm O.D., Strategic applications Inc.) was inserted through the drilled hole and fixed by optical adhesive (NOA81, Norland products) and cured under UV light. Silastic tubing (VWR scientific products) or PTFE tubing (Plasmatech Co.) with inner diameters D=3.175 mm were connected to the tee union tube fitting using a connector and adaptor (IDEX Health & Science).

FIG. 1 shows schematic illustration of the coaxial turbulent jet mixer for high-throughput synthesis of nanoparticles. Schematic illustration of the coaxial turbulent jet mixer with turbulence induced by vortex (FIG. 1A) and turbulence induced by jetting (FIG. 1B). The insets show the top view of the turbulent jets when R=0.3 and Re=353 (FIG. 1A), and when R=10 and Re=1311 (FIG. 1B).

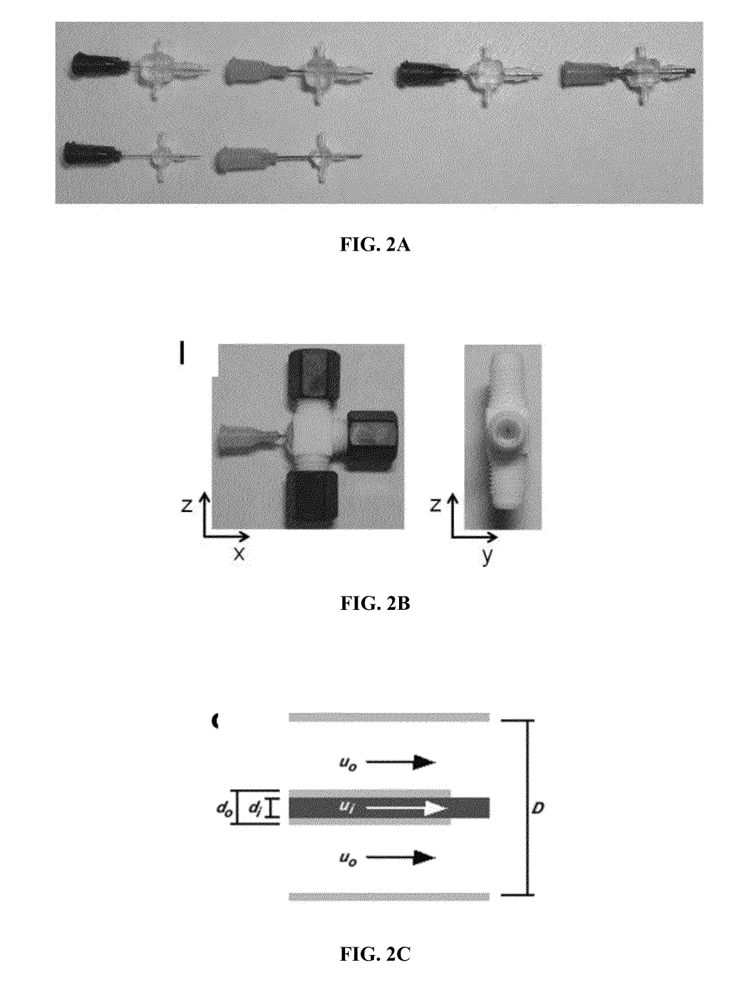

The coaxial turbulent jet mixer is prepared by inserting a syringe needle into a “T” tube fitting. Fabrication can be accomplished within 30 min without requiring specialized equipment, micro-fabrication facilities, or specialized skills. A wide choice of standard fittings and materials can be used to construct the coaxial turbulent jet mixer. FIGS. 2A and 2B show syringe needles with various sizes (i.e., 30 G, 23 G, 19 G, and 15G) and tee union tube fittings made of clear polycarbonate and polytetrafluoroethylene (PTFE) that can be used for the purpose. In addition, the coaxial turbulent jet mixer is reusable and the needle is easily replaceable as needed. While a wide choice of standard fittings and materials can be used to construct the cozxial turbulent juet mixer, in the examples below, PTFE fittings and tubing compatible with a variety of solvents are used.

FIG. 2A shows coaxial turbulent jet mixer made from syringe needles (i.e., 30 G, 23 G, 19 G, and 15 G) and clear polycarbonate tee union tube fittings (i.e., ⅛″ and 1/16″). FIG. 2B shows coaxial turbulent jet mixer made from 23G needle and ⅛″ PTFE tee union tube fittings. FIG. 2C shows schematic drawing of geometry and flow condition of the coaxial turbulent jet mixer.

### Characterization of Mixing in the Coaxial Turbulent Jet Mixer

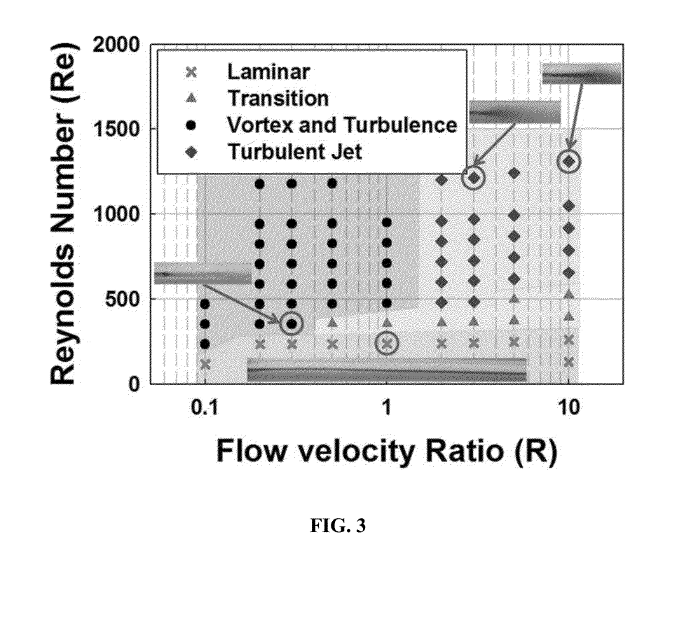

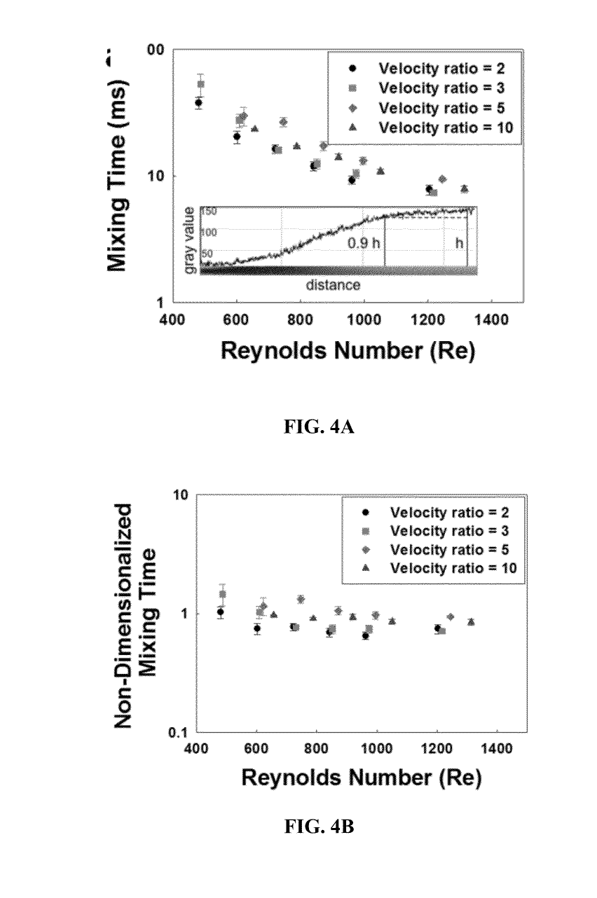

Since the mixing behavior is strongly influenced by the flow condition in a fixed geometry, the flow in a coaxial turbulent jet mixer was examined by changing the flow condition systematically. The flow rates were controlled by syringe pumps during the operation of coaxial turbulent jet mixer. The tee union made of clear polycarbonate and transparent silastic tubes were used for the imaging and characterization of flow behavior in the coaxial turbulent jet mixer, since PTFE tee union and Teflon tubes are not transparent (FIGS. 2A and 2B). The flow rates were controlled by syringe pumps (Harvard Apparatus). Mixing of the inner and outer streams was visualized using phenolphthalein (Sigma-Aldrich), a pH indicator that changes color from pink to colorless as it goes from basic to neutral or acidic environments. Because phenolphthalein is insoluble in water, 0.1 N sodium hydroxide (Sigma-Aldrich) in water-ethanol mixture (1:2 in volume ratio) with 1% w/v phenolphthalein was used as pink colored basic inner solution. The composition of outer solution was 0.1 N hydrogen chloride (Sigma-Aldrich) in water-ethanol mixture (1:2 in volume ratio). To match the densities of inner fluid and outer fluid, same composition of water-ethanol mixture was used to prepare basic inner solution and acidic outer solution. As a result, the vertical drift is negligible compared to the horizontal flow (FIGS. 1 and 3). As mixing occurred in the coaxial turbulent jet mixer the color of liquid jet changed from pink to colorless, because the acidic outer solution neutralized or acidified the basic inner solution. Here, the color of fluid indicated the degree of mixing. The ν of water-ethanol mixture is calculated by using the previously reported values of dynamic viscosity (μ) and density (ρ) in water-ethanol mixture. See, Khattab, I., Bandarkar, F., Fakhree, M. & Jouyban, A. Density, viscosity, and surface tension of water+ethanol mixtures from 293 to 323K. Korean J. Chem. Eng. 29, 812-817 (2012), which is incorporated by reference in its entirety. Enhanced aluminum coated right angle prism mirror (Edmund optics Inc.) was placed next to the coaxial turbulent jet mixer to capture top and side views simultaneously, when the images and videos were taken from above the coaxial turbulent jet mixer. The mixing in the coaxial turbulent jet mixer was captured as images and videos by systematically varying R and Re. L was determined by analyzing images in Image J. The L was defined as the length at which the gray value is 90% of the intensity difference between the completely mixed flow far downstream along the centerline and the tip of the inner syringe needle (inset of FIG. 4A). In case the flow was still unmixed at the edge of right angle prism mirror (i.e., in 75 mm), L was not estimated.

Understanding the flow behavior and mixing time (τₘᵢₓ) in the coaxial turbulent jet mixer is important because NP assembly is strongly influenced by τₘᵢₓ. See, Johnson, B. K.; Prud'homme, R. K. Mechanism for Rapid Self-Assembly of Block Copolymer Nanoparticles. Phys. Rev. Lett. 2003, 91, 118302, and Karnik, R.; Gu, F.; Basto, P.; Cannizzaro, C.; Dean, L.; Kyei-Manu, W.; Langer, R.; Farokhzad, 0. C. Microfluidic Platform for Controlled Synthesis of Polymeric Nanoparticles. Nano Lett. 2008, 8, 2906-2912, each of which is incorporated by reference in its entirety. The mixing behavior in the coaxial turbulent jet mixer was characterized by changing the two dimensionless parameters of flow velocity ratio (R) and average Re, which can be defined by equation (1) and (2), respectively.

$$\begin{matrix}R = \frac{{u}_{i}}{{u}_{o}} \\ and & ( 1 ) \\ Re = \frac{QD}{vA} & ( 2 )\end{matrix}$$

Where uᵢ and uₒ are input velocities of inner stream and outer stream, respectively. Q and ν are total flow rate and the kinematic viscosity of the fluid mixture. D and A are the diameter and cross-sectional area of outer tube, respectively. The ν of water-ethanol mixture is calculated by using the previously reported values of dynamic viscosity (μ) and density (ρ) in water-ethanol mixture. See, Baldyga, J. & Bourne, J. R. Turbulent mixing and chemical reactions. (John Wiley & Sons, 1999), and Khattab, I., Bandarkar, F., Fakhree, M. & Jouyban, A. Density, viscosity, and surface tension of water+ethanol mixtures from 293 to 323K. Korean J. Chem. Eng. 29, 812-817 (2012), each of which is incorporated by reference in its entirety. Since the geometry of device (i.e., D and A) and composition of inner and outer fluid (i.e., μ, ρ, and ν) were fixed, volumetric flow rates of inner (Qᵢ) and outer fluid (Qₒ) were controlled by using syringe pumps to change the Re and R. The relations between input velocities (uᵢ and uₒ) and volumetric flow rate of inner and outer stream (Qᵢ and Qₒ) are defined by equation (3) and (4), respectively.

$$\begin{matrix}{u}_{i} = \frac{4 {Q}_{i}}{π {d}_{i}^{2}} & ( 3 ) \\ {u}_{o} = \frac{4 {Q}_{o}}{π ( {D}^{2} - {d}_{o}^{2} )} & ( 4 )\end{matrix}$$

Where dᵢ and dₒ are the inner diameter and outer diameter of syringe needle.

The coaxial turbulent jet mixer can be made from standard fittings and materials that exhibit good solvent resistance. The fluid flow and mixing process does not require very precise alignment of the inner and outer tubes. To ensure that the inner tube is coaxially aligned with the outer tube (typically within 0.5 mm of the axis of the outer tube), metal syringe needle can be used as an inner tube and fixed the outer flexible tubing on a rigid plate. By analyzing the images and movie clips of visualized jet flow, the flow behavior could be categorized to laminar, transition, vortex and turbulence, and turbulent jet regimes as summarized in the phase diagram (FIG. 3). If there are significant differences in relative densities and viscosities of the inner and outer fluid, the inner stream can touch the inner wall of outer tube in the laminar flow regime. In the vortex and turbulence regime and turbulent jet regime, on the other hand, this effect is negligible because of fast lateral flow velocity of fluids. Since the coaxial turbulent jet mixer was operated in the turbulent jet regime for NP synthesis, the effect of small differences in relative densities and viscosities on NP synthesis is negligible. By flowing NP precursor and non-solvent through inner and outer tubes, respectively, the ratio of NP precursor and non-solvent can be fixed, which is one of the important parameters that determines the NP size distribution. In laminar flow regime, the inner flow is focused by outer flow and stable stratified flow is maintained. Because slow diffusional mixing is dominant in the laminar flow regime, the fluid is not completely mixed. In transition regime, the flow is unstable and unexpectedly turns from laminar flow to turbulent flow, and vice versa. In vortex and turbulence regime, the micro-vortex is generated in outer flow and turbulence is developed at the tip of focused inner flow by the micro-vortex. The focused inner flow is not distinguishable at small R (R=0.1) and large Re (Re>500) because the color of focused inner stream changed from pink to colorless in tens of micrometer. In turbulent jet regime, inner stream is spurted out as a turbulent jet.

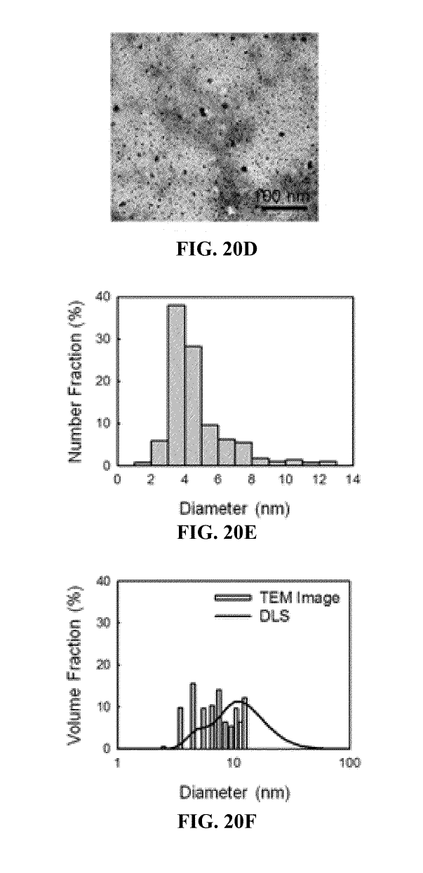

Fluid flow and mixing in the device was visualized using phenolphthalein, a pH indicator that changes color from pink to colorless as it goes from basic to acidic environments (FIG. 1 insets and FIG. 19). With a basic (pink) solution of phenolphthalein as the inner fluid and an acidic outer fluid stream, the flow behavior could be categorized to laminar, transition, vortex and turbulence, and turbulent jet regimes as summarized in the phase diagram (FIG. 3) obtained by analyzing the flow images (FIG. 1 insets and FIG. 3) and videos. When R<1, the inner fluid is focused by the outer fluid and the flow rate difference at the tip of the needle creates recirculating vortices at high Re. Mixing time is difficult to quantify in this regime due to entrainment of the fluid in vortices and the flow rate of the inner fluid is low (Qᵢ≦0.01 Qₒ), making this regime less desirable for nanoparticle synthesis. This regime transitions to vortices and turbulence at higher Re, where a micro-vortex is generated at the tip of the needle and turbulence is developed at the tip of focused inner flow by the micro-vortex. At higher velocity ratios (R≧1), the flow remains laminar at low Re and a stable stratified flow is maintained. Because slow diffusional mixing is dominant in the laminar flow regime, the fluid is not completely mixed. As the Re increases, the flow becomes unstable and fluctuates between laminar and turbulent flows in the transition regime, and finally transitions to a completely turbulent jet regime.

A mixing length (L) was determined from images of the device taken under different flow conditions of Re and R. Since the phenolphthalein appears pink, the complementary green channel of the RGB images was analyzed in ImageJ to extract a L from each images (inset of FIG. 4A). The intensity profile of the green color channel along the centerline of the jet was found, and the mixing length L was defined as the length at which the difference between it and the beginning of the jet was 90% of the intensity difference between the completely mixed flow far downstream and the beginning of the jet.

In case of vortex and turbulence regime and turbulent jet regime, the mixing length (L) can be estimated as a function of Re and R. L is defined as the length at which the gray value of the phenolphthalein color is 90% of the intensity difference between the completely mixed region far downstream along the centerline and the tip of the syringe needle (inset of FIG. 4A). In case of laminar regime, L could not be estimated because complete mixing was not achieved. In case of transition regime, L also could not be estimated because the flow was unmixed or unstable. To estimate the τₘᵢₓ in a simple way, it is assumed that the mixed fluid flow at u_{avg} throughout the mixing process, which can be defined by equation (5).

$${u}_{avg} = \frac{( {D}^{2} - {d}_{o}^{2} ) {u}_{o} + {d}_{i}^{2} {u}_{i}}{{D}^{2}} = \frac{4 ( {Q}_{i} + {Q}_{o} )}{π {D}^{2}} \qquad ( 5 )$$

Although uᵢ and uₒ are different, the speed of mixed fluid will eventually reach average velocity (u_{avg}), which is defined by equation (5). Under the assumption, τₘᵢₓ can be estimated as

$${τ}_{mix} = \frac{L}{{u}_{avg}} \qquad ( 6 )$$

Operating in turbulent jet regime, the τₘᵢₓ was tunable in the range of 7-53 ms by changing the Re, with faster mixing at higher Re (FIG. 4A).

To theoretically capture the mixing timescale, the engulfment, deformation, diffusion (EDD) turbulent micromixing model is used. The unmixed fluid first enters turbulent vortices that stir the fluid at their characteristic frequency, leading to folding of the fluids into a layered structure. The layers become thinner with time, and molecular diffusion finishes the mixing process once the layers are thin enough. See, Baldyga, J. & Bourne, J. R. Turbulent mixing and chemical reactions. (John Wiley & Sons, 1999), which is incorporated by reference in its entirety. The characteristic timescale (τ_{ω}) in EDD turbulent micromixing model is given by

$${τ}_{ω} \approx 12.7 {( \frac{v}{〈 ɛ 〉} )}^{0.5} \qquad ( 7 )$$

where [symbol] ε [symbol] is the average turbulent kinetic energy dissipation rate in the core of a pipe flow, given by

$$〈 ɛ 〉 = 0.0668 \frac{{u}_{avg}^{3}}{{Re}^{0.25} D} \qquad ( 8 )$$

Normalizing τₘᵢₓ by τ_{ω} collapses the mixing time to a value of unity independent of the Re (FIG. 4B), demonstrating that the mixing time can be predicted by the EDD turbulent micromixing model when the coaxial turbulent jet mixer is operated in turbulent jet regime.

### Preparation of Various Nanoparticles

### 1. Preparation of PLGA-PEG Nanoparticles

The PLGA_{45K}-PEG_{5K} and PLGA_{95K}-PEG_{5K} (Boehringer Ingelheim GmbH) was dissolved in acetonitrile (ACN, Sigma-Aldrich) at concentrations of 10 or 50 mg/mL. For the drug loading test, docetaxel (LC laboratories) and Human recombinant insulin (Sigma-Aldrich) were used as model therapeutic agents. The PLGA-PEG precursor in ACN and deionized water were used as inner and outer stream, respectively. To make insulin loaded PLGA-PEG nanoparticles, dimethyl sulfoxide (DMSO, Sigma-Aldrich) was used as organic solvent, because insulin is not soluble to ACN. During the nanoparticle synthesis, the flow rates were controlled by syringe pumps (Harvard Apparatus). To estimate the Re, ν of water-ACN mixture and water-DMSO mixture are calculated by using the previously reported values of μ and ρ in water-ACN mixture and water-DMSO mixture, respectively. See, Cunningham, G. P., Vidulich, G. A. & Kay, R. L. Several properties of acetonitrile-water, acetonitrile-methanol, and ethylene carbonate-water systems. J. Chem. Eng. Data 12, 336-337 (1967), and LeBel, R. G. & Goring, D. A. I. Density, viscosity, refractive index, and hygroscopicity of mixtures of water and dimethyl sulfoxide. J. Chem. Eng. Data 7, 100-101 (1962), each of which is incorporated by reference in its entirety. The resulting nanoparticle suspensions were purified by ultrafiltration using Amicon Ultracel 100 K membrane filters. In case of bulk synthesis, 100 μL of polymeric precursor solution was mixed drop-wise with 1 mL of water for about 2 h under magnetic stirring.

### 2. Preparation of Lipid Vesicles

Dimyristoylphosphatidylcholine (DMPC, Avanti Polar Lipids Inc.), cholesterol (Avanti Polar Lipids Inc.), and dihexadecyl phosphate (DCP, Sigma-Aldrich) in a molar ratio of 5:4:1 were dissolved in chloroform (Sigma-Aldrich). The chloroform was removed by evaporation under a stream of nitrogen gas at 30° C. The glass vial with a dry lipid blend was stored in a desiccator for 24 h to remove residual chloroform. The lipid blend was dissolved in isopropyl alcohol (IPA) at concentration of 5 mM. To make fluorescent lipid vesicles, 1 wt % of 1,1′-Dioctadecyl-3,3,3′,3′-tetramethylindocarbocyanine perchlorate (DiIC18, Sigma-Aldrich) with respect to the total weight of the lipid blend was added to the lipid blend in the IPA solution. The lipid blend in IPA solution and phosphate buffered saline (PBS) were used as inner and outer stream, respectively. To estimate the Re, ν of water-IPA mixture is calculated by using the previously reported values of μ and ρ in water-IPA mixture. See, Lebo, R. B. Properties of mixtures of isopropyl alcohol and water. J. Am. Chem. Soc. 43, 1005-1011 (1921), which is incorporated by reference in its entirety. In case of the bulk synthesis, 100 μL of lipid blend in IPA solution was mixed drop-wise with 1 mL of PBS for about 2 h under magnetic stirring.

### 3. Preparation of Iron Oxide Nanoparticles

The iron (II) chloride tetrahydrate and iron (III) chloride (Sigma-Aldrich) in a molar ratio Fe (II)/Fe (III) of 1:1 was dissolved in 1 N hydrochloric acid (Sigma-Aldrich) at concentration of 10 mM. The iron oxide precursor in hydrochloric acid and alkaline solution of tetramethylammonium hydroxide (TMAOH, Sigma-Aldrich) at concentration of 172 mM were used as inner and outer stream, respectively. In case of bulk synthesis, 100 μL of iron oxide precursor in hydrochloric acid solution was mixed drop-wise with 1 mL of TMAOH for about 2 h under magnetic stirring.

### 4. Preparation of Polystyrene Nanoparticles

Polystyrene (MW 35000, Sigma-Aldrich) was dissolved in tetrahydrofuran (THF, Sigma-Aldrich) at a concentration of 1 mg/mL. To make fluorescent polystyrene nanoparticles, 10 wt % of perylene (Sigma-Aldrich) to the total weight of polystyrene was added to the polystyrene in THF solution. The polystyrene precursor in THF and deionized water were used as inner and outer stream, respectively. To estimate the Re, ν of water-THF mixture is calculated by using the previously reported values of μ and ρ in water-THF mixture. See, Pinder, K. L. Viscosity of the tetrahydrofuran-water system. Can. J. Chem. Eng. 43, 274-275 (1965), which is incorporated by reference in its entirety. In case of bulk synthesis, 100 μL of polystyrene precursor solution was mixed drop-wise with 1 mL of water for about 2 h under magnetic stirring.

5. Preparation of siRNA/PEI Polyplex Nanoparticles

The siRNA/PEI polyplex nanoparticles were prepared by mixing aqueous solution of siRNA (Luciferase (GL3): sequence 5′-CUU ACG CUG AGU ACU UCG AdTdT-3′ (SEQ ID NO: 1) (sense) and 5′-UCG AAG UAC UCA GCG UAA GdTdT-3′ (SEQ ID NO: 2) (antisense)) and Polyethyleneimine (PEI_{25K, Branched}) at varying molar ratios (siRNA:PEI=1:1 to 1:4). The aqueous siRNA solution and aqueous PEI solution were used as inner and outer stream, respectively. In case of bulk synthesis, 4 mL of aqueous siRNA solution and 40 mL of aqueous PEI solution was mixed by votexing at 1500 rpm, at different concentrations so as to keep the desired molar ratios of siRNA and PEI as described above. The siRNA/PEI polyplex nanoparticles were lyophilized and stored at −20° C. until use.

### 6. Characterization of Nanoparticles

The size distributions by volume fraction of synthesized nanoparticles and polystyrene microspheres (99 nm, Bangs laboratories Inc.) were measured using dynamic light scattering with Zetasizer Nano ZS (Malvern Instruments Ltd.). The synthesized nanoparticles were imaged by TEM (JEOL 200CX). For TEM imaging, PLGA-PEG nanoparticles and lipid vesicles were stained by uranyl acetate (Electron Microscopy Sciences). The amounts of docetaxel loading in the PLGA-PEG nanoparticles were measured by HPLC (Agilent Technologies, 1100 Series) using established procedures. See, Cheng, J. et al. Formulation of functionalized PLGA-PEG nanoparticles for in vivo targeted drug delivery. Biomaterials 28, 869-876 (2007), which is incorporated by reference in its entirety. The amount of insulin loading in the PLGA-PEG nanoparticles were measured by a protein bicinchoninic acid (BCA) assay (Lamda Biotech). Fluorescent images of lipid vesicles and polystyrene nanoparticles were acquired using an epi-fluorescence microscope (Eclipse TE 2000-U, Nikon). The typical sample volume for characterization was 15 mL and 1.1 mL for coaxial turbulent jet mixer and bulk synthesis method, respectively.

### 7. In Vitro Transfection

All the in vitro transfection experiments were performed in quadruplicate. Dual-Luciferase (Luc) HeLa cells were grown and then seeded in 96-well plates at a density of 10,000 cell/well 18 h before transfection. The cells were incubated for 24 h with various amounts of siRNA/PEI polyplex nanoparticles in media without FBS. For all the nanoparticle treatments encapsulating GL3 siRNA, scrambled siRNA/PEI polyplex nanoparticles were used as negative control. Lipo2000/siRNA complex was formulated following the manufacturer's protocol (Invitrogen) and was used as a positive control for transfection. After the incubation period, the cells were washed with growth media and allowed to grow for a period of 24 h. The HeLa cells were then analyzed for expression of firefly and renilla luciferase signals by using the Dual-Glo™ Luciferase Assay System (Promega). The luminescence intensity was measured using a microplate reader (BioTek).

### 8. Results

### Flow Regimes and Mixing Timescale

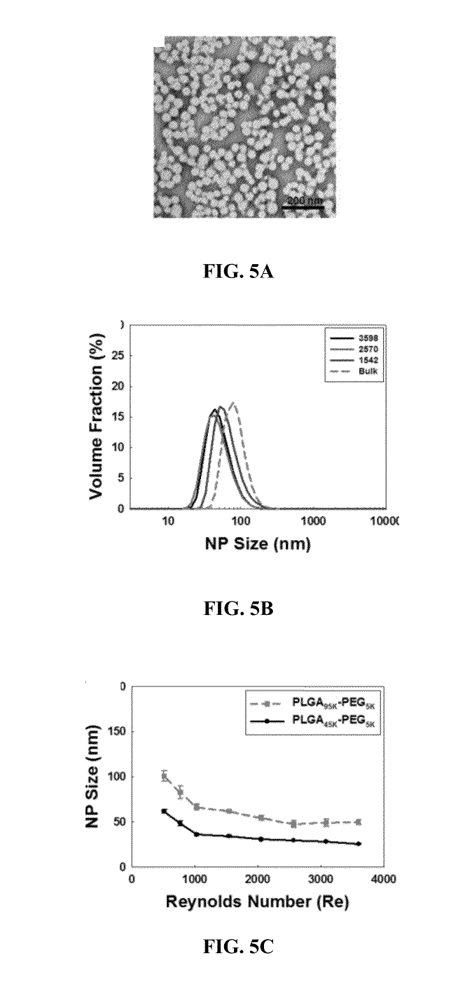

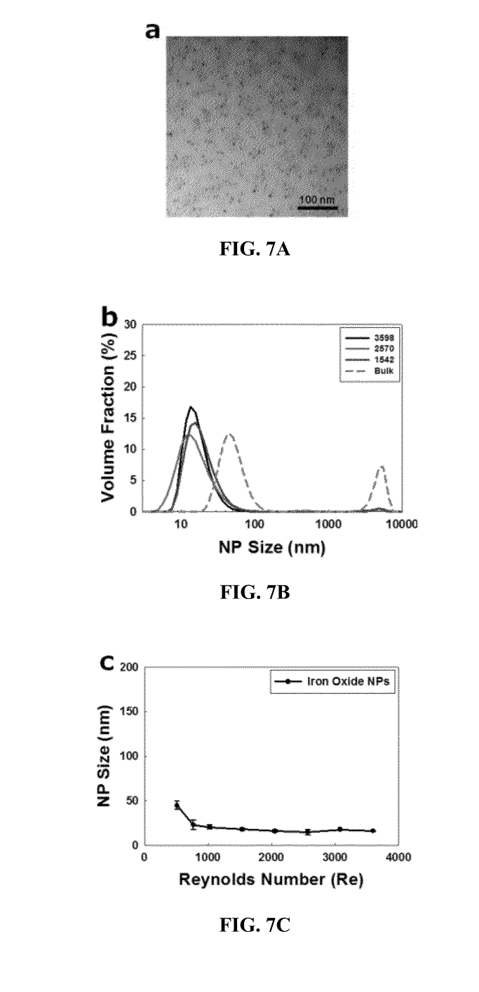

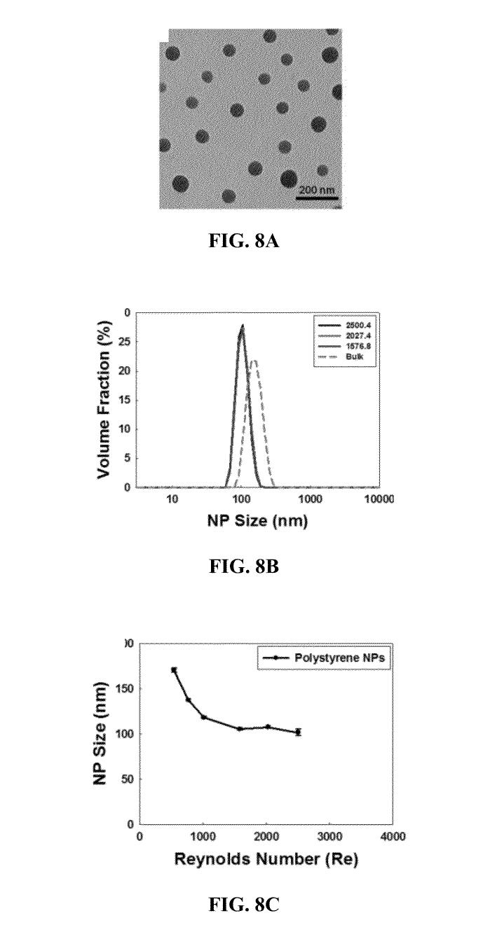

The coaxial turbulent jet mixer is a versatile system that can synthesize various types of nanoparticles by rapid mixing/nanoprecipitation in a controlled and high-throughput manner. As conventional PDMS microfluidic devices are not compatible with most organic solvents, the choice of nanoparticle precursor solutions is limited. However, the coaxial turbulent jet mixer can be easily fabricated from PTFE (FIG. 2B) that has excellent compatibility with organic solvents. As a proof of concept, the preparation of various type of nanoparticles that are widely used for biomedical applications are demonstrated, including PLGA-PEG nanoparticles, lipid vesicles, iron oxide nanoparticles, polystyrene nanoparticles, and siRNA/PEI polyplex nanoparticles by using the coaxial turbulent jet mixer (FIGS. 5-8, and 13).

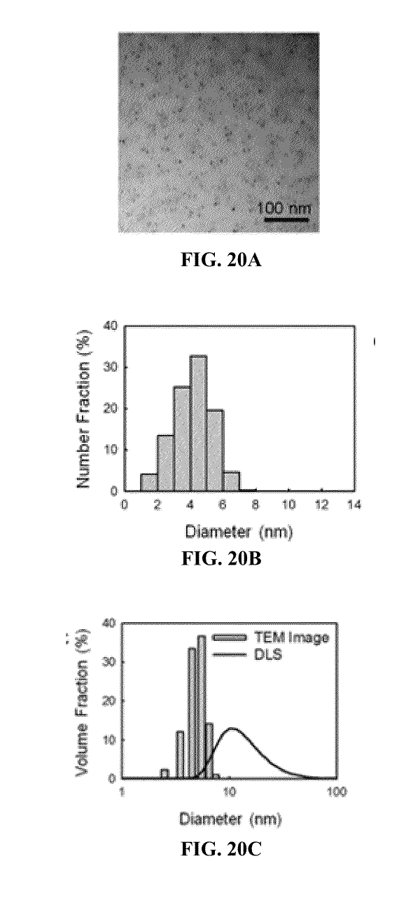

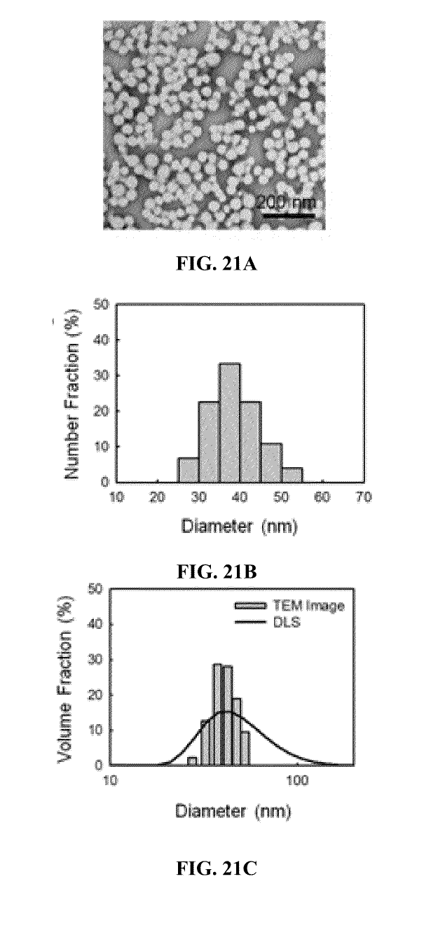

PLGA-PEG nanoparticles, lipid vesicles, iron oxide nanoparticles, polystyrene nanoparticles, and siRNA/PEI polyplex nanoparticles were synthesized by nanoprecipitation by injecting nanoparticles precursors into the stream of non-solvent in 1:10 volumetric flow rate ratio (R=8.514) (FIG. 1). In case of PLGA-PEG nanoparticles, production rates up to 2.19 g/min (˜3.15 kg/d) were achieved when the coaxial turbulent jet mixer was operated at high Re and high polymer concentration (i.e., 50 mg/mL) (Table 1). The synthesized nanoparticles were uniform in size as confirmed in both transmission electron microscope (TEM) images (FIGS. 5A, 6A, 7A, 8A, and 19) and by dynamic light scattering (FIGS. 5B, 6B, 7B, 8B, and 19). The diameter obtained from dynamic light scattering is consistent with that obtained from TEM images in the case of PLGA-PEG NPs. Dynamic light scattering yields a larger diameter for iron oxide NPs due to dipole-dipole interactions (see, Lim, J.; Yeap, S.; Che, H.; Low, S. Characterization of Magnetic Nanoparticle by Dynamic Light Scattering. Nanoscale Res. Lett. 2013, 8, 381, which is incorporated by reference in its entirety), but even in this case TEM analysis reveals a tighter distribution of NP sizes compared to bulk synthesis (FIGS. 20A-20F). The size distributions of nanoparticles prepared by coaxial turbulent jet mixer were more uniform compared to that of nanoparticles prepared by conventional bulk synthesis methods (FIGS. 5B, 6B, 7B and 8B) and τₘᵢₓ is smaller than the characteristic aggregation timescale (τ_{agg}), consistent with previous reports of nanoporecipitation using microfluidic devices. See, Karnik, R. et al. Microfluidic platform for controlled synthesis of polymeric nanoparticles. Nano Lett. 8, 2906-2912 (2008), and Rhee, M. et al. Synthesis of size-tunable polymeric nanoparticles enabled by 3D hydrodynamic flow focusing in single-layer microchannels. Adv. Mater. 23, H79-H83 (2011), each of which is incorporated by reference in its entirety. FIG. 18 shows that the nanoparticles prepared by coaxial turbulent jet mixer are more uniform in their size distribution compared to the commercially available nanoparticles synthesized by emulsion polymerization (purchased from Bangs Laboratories, Inc.).

TABLE 1
Total flow rate of fluids and production rate of PLGA-PEG nanoparticles
in a coaxial turbulent jet mixer with different Re. Here, the volumetric
flow rate ratio of inner flow to outerflow was fixed at 0.1.

| Reynolds number | 1542 | 2570 | 359 |
|---|---|---|---|
| Total flow rate | 206.25 mL/ | 343.75 mL/ | 481.25 mL/ |
| | min | min | min |
| Production rate | 0.1875 g/ | 0.3125 g/ | 0.4375 g/ |
| (10 mg/mL) | min | min | min |
| Production rate | 0.9375 g/ | 1.5625 g/ | 2.1875 g/ |
| (50 mg/mL) | min | min | min |

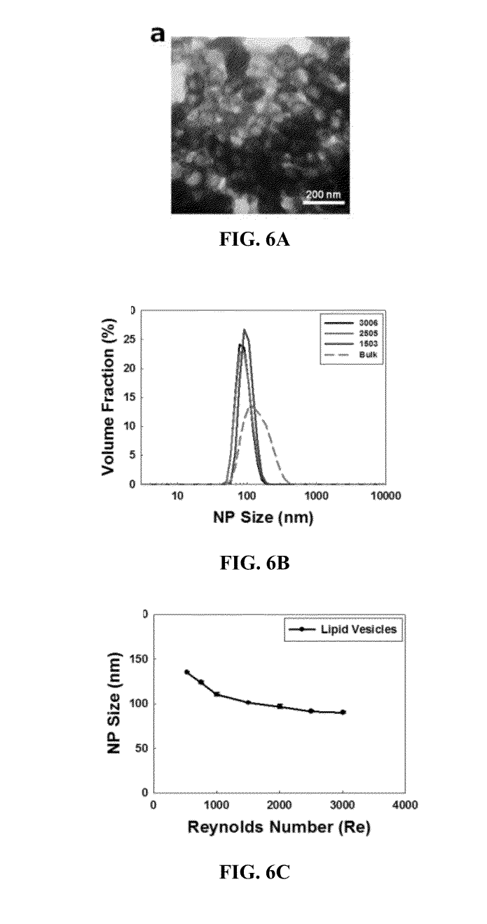

Using the coaxial turbulent jet mixer, the size of the nanoparticles could be precisely controlled simply by changing Re for given nanoparticle precursor solutions (FIGS. 5C, 6C, 7C and 8C), since τₘᵢₓ is precisely controllable by changing Re (FIG. 4A). Nanoparticles obtained using the coaxial turbulent jet mixer are smaller than those synthesized by the bulk synthesis method because the τₘᵢₓ is smaller than the characteristic aggregation time scale (τ_{agg}) (FIGS. 5B, 6B, 7B and 8B). See, Johnson, B. K. & Prud'homme, R. K. Mechanism for rapid self-assembly of block copolymer nanoparticles. Phys. Rev. Lett. 91, 118302 (2003), and Karnik, R. et al. Microfluidic platform for controlled synthesis of polymeric nanoparticles. Nano Lett. 8, 2906-2912 (2008), each of which is incorporated by reference in its entirety. In the current design of coaxial turbulent jet mixer, the size of nanoparticles could be controlled precisely and reproducibly in the range of 25-60 nm and 50-100 nm by simply changing Re, when PLGA_{45K}-PEG_{5K} and PLGA_{95K}-PEG_{5K} at a concentration of 10 mg/mL were used as polymeric precursors, respectively (FIG. 5C). Similar to the case of PLGA-PEG nanoparticles, the size of lipid vesicles, iron oxide nanoparticles, and polystyrene nanoparticles could be controlled precisely and reproducibly by simply changing Re (FIGS. 6C, 7C and 8C). The influence of τₘᵢₓ on the size of nanoparticles disappears at a certain Re, suggesting that these nanoparticles have reached the size corresponding to the limit of rapid mixing.

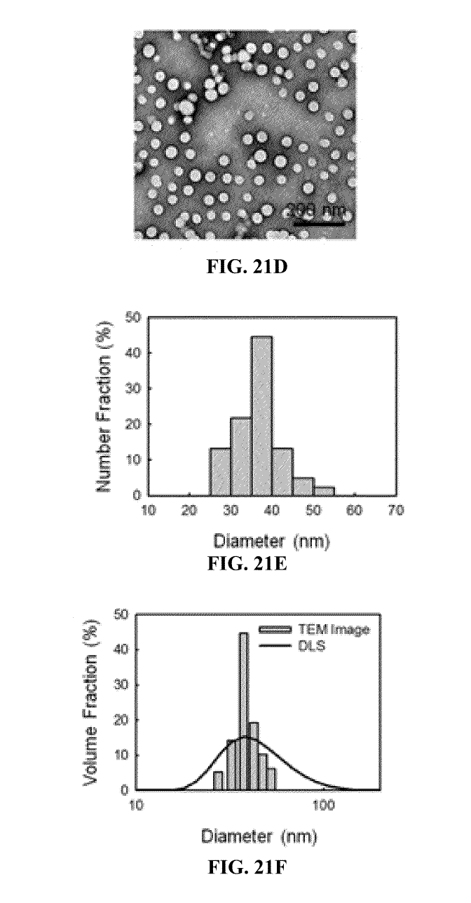

The coaxial turbulent jet mixer provides an inherently high NP production throughput due to operation in the turbulent regime with device dimensions in the millimeter scale, which involves high Re and high flow rates. Typical Re in the coaxial turbulent jet mixer ranged from 500 to 3500 (FIGS. 5C, 6C, 7C, 8C and Table 1). Assuming that all the NP precursors flowing into the device are essentially converted to NPs (see, Lim, J.-M.; Bertrand, N.; Valencia, P. M.; Rhee, M.; Langer, R.; Jon, S.; Farokhzad, O. C.; Karnik, R. Parallel Microfluidic Synthesis of Size-Tunable Polymeric Nanoparticles Using 3D Flow Focusing towards in vivo Study. Nanomedicine 2014, 10, 401-409, which is incorporated by reference in its entirety), in case of PLGA-PEG NPs production rates were estimated up to 2.19 g/min (˜3.15 kg/d) when the coaxial turbulent jet mixer is operated at high Re (i.e., Re=3598) and high polymer concentrations (i.e., 50 mg/mL) (Table 1). Since the coaxial turbulent jet mixer operates in continuous mode, the quality of NPs can be independent of batch size. To examine the effect of aggregation of precipitates on NP synthesis, PLGA₉₅ₖ-PEG₅ₖ NPs are prepared where the high molecular weight of hydrophobic PLGA block tends to promote aggregate on the channel walls. See, Rhee, M.; Valencia, P. M.; Rodriguez, M. I.; Langer, R.; Farokhzad, O. C.; Karnik, R. Synthesis of Size-Tunable Polymeric Nanoparticles Enabled by 3D Hydrodynamic Flow Focusing in Single-Layer Microchannels. Adv. Mater. 2011, 23, H79-H83, which is incorporated by reference in its entirety. The size distribution of PLGA₉₅ₖ-PEG₅ₖ NPs was essentially identical within standard error for the tens of milligram synthesis scale and for a few gram scale (FIGS. 21A-21F), illustrating the robustness of the coaxial turbulent jet mixer.

In FIGS. 21A-21F, the PLGA₉₅ₖ-PEG₅ₖ was dissolved in acetonitrile at concentration of 10 mg/mL. The flow rates of PLGA-PEG precursor and deionized water were 31.25 mL/min and 312.5 mL/min to achieve Re of 2570, respectively. To make PLGA-PEG NPs at the tens of milligram scale, the output was collected from the coaxial turbulent jet mixer for 3 to 5 s after waiting for ˜3 s to reach steady state. To make PLGA-PEG NPs at the few gram scale, the output was collected from the coaxial turbulent jet mixer for ˜5 min while refilling the syringes as needed. The size distribution of PLGA₉₅ₖ-PEG₅ₖ NPs prepared using coaxial turbulent jet mixer for the tens of milligram synthesis scale and for a few gram scale was essentially identical within standard error.

### Encapsulation of Functional Agents

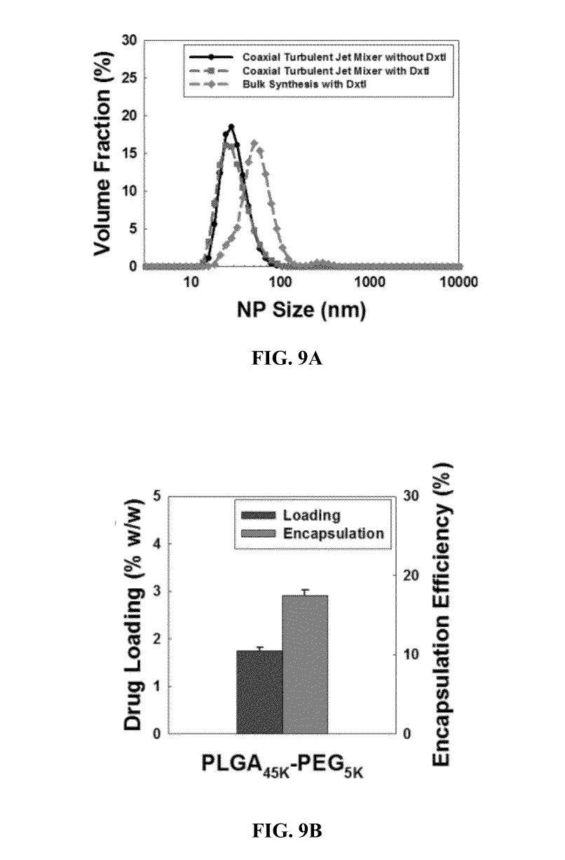

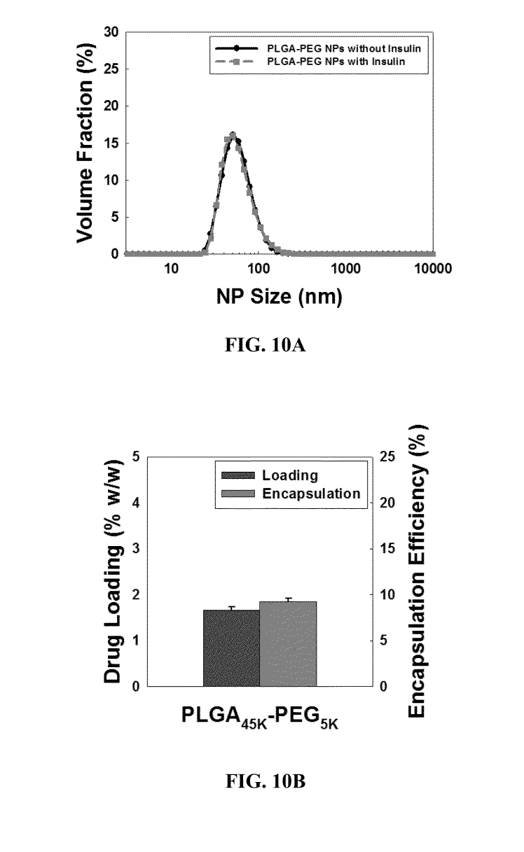

PLGA-PEG nanoparticle have received considerable attention in the field of drug delivery, because they are biodegradable and biocompatible, have the ability to incorporate drug molecules, and can controllably release the drug molecules. See, Farokhzad, O. C. et al. Nanoparticle-aptamer bioconjugates: a new approach for targeting prostate cancer cells. Cancer Res. 64, 7668-7672 (2004), and Farokhzad, O. C. & Langer, R. Impact of nanotechnology on drug delivery. ACS Nano 3, 16-20 (2009), each of which is incorporated by reference in its entirety. To further assess to potential of the PLGA-PEG nanoparticle platform for drug delivery applications, drug molecules were loaded in the PLGA-PEG nanoparticles. FIG. 9A shows the size distribution of PLGA-PEG nanoparticles prepared by using PLGA_{45K}-PEG_{5K} as the polymeric precursor and docetaxel as a model therapeutic agent. When PLGA-PEG nanoparticles were assembled by coaxial turbulent jet mixer, the size distribution of nanoparticles is uniform both without and with adding a model therapeutic agent of docetaxel. The drug loading, defined as the mass fraction of drug molecule in the nanoparticles, and the encapsulation efficiency defined as the fraction of initial drug encapsulated in the nanoparticles, were shown in FIG. 9B. Similarly, insulin could be associated to PLGA-PEG nanoparticles. FIG. 10A shows the size distribution of PLGA-PEG nanoparticles prepared by using PLGA_{45K}-PEG_{5K} as the polymeric precursor and insulin as a model therapeutic agent. Here, dimethyl sulfoxide (DMSO) was used as organic solvent, because insulin is not soluble to ACN. The drug loading and the encapsulation efficiency were shown in FIG. 10B.

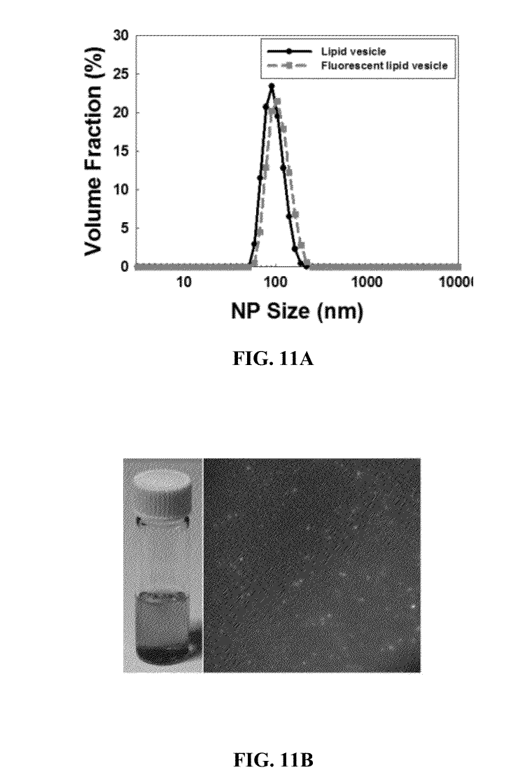

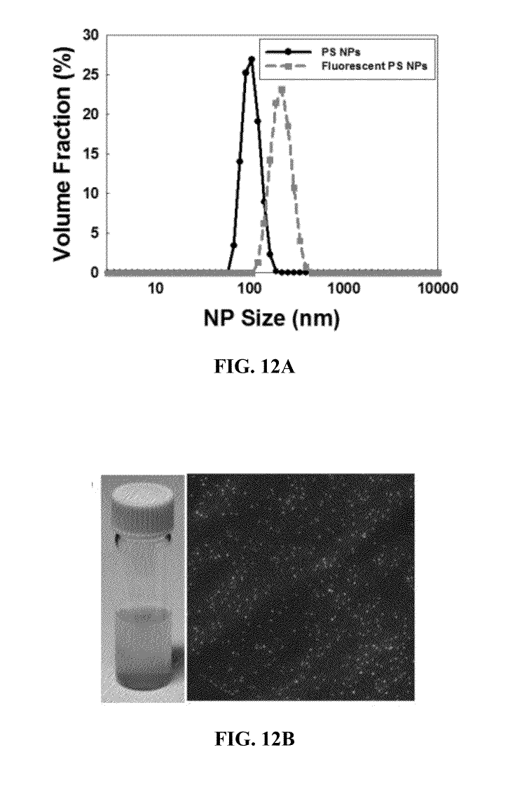

Fluorescent nanoparticles have received considerable attention in cancer imaging in recent years because of their improved sensitivity and photostability compared to the traditional fluorescent dyes and fluorescent proteins. In addition, fluorescent nanoparticles with certain size range can be passively targeted to tumor tissue by enhanced permeability and retention (EPR) effect. To further assess to potential of the lipid vesicle and polystyrene nanoparticle platform for biomedical imaging applications, fluorescent dyes were loaded in the lipid vesicle and polystyrene nanoparticles. Because membrane-intercalating fluorescent dye was used to assemble fluorescent lipid vesicles, the average size of lipid vesicles is slightly increased from 97 nm to 111 nm while maintaining the uniform size distribution (FIG. 11A). FIG. 11B shows the digital camera image and fluorescent microscope images of fluorescent lipid vesicles. In case of hydrophobic dye is encapsulated in the polystyrene nanoparticles, the average size is increased from 105 nm to 224 nm (FIG. 12A). FIG. 12B shows the digital camera image and fluorescent microscope images of fluorescent polystyrene nanoparticle.

In vitro Gene Knockdown by siRNA/PEI Polyplex Nanoparticles

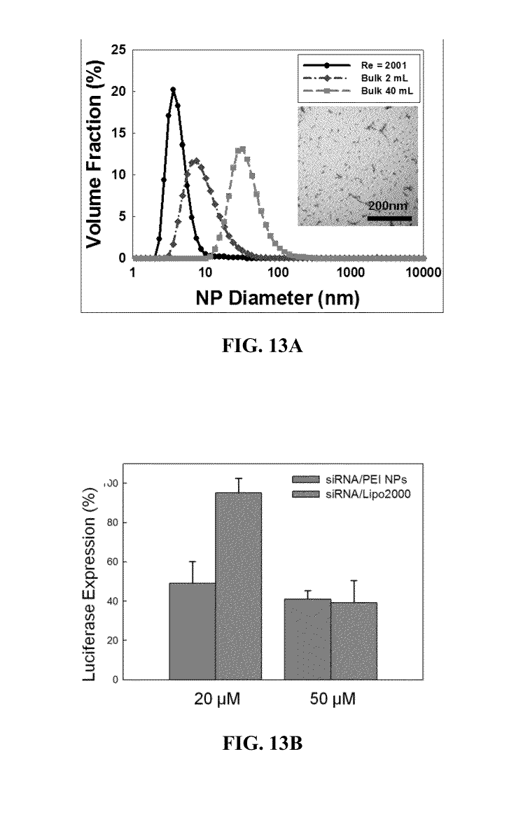

There are growing interests in exploring RNAi therapy for the non-druggable targets and siRNA has emerged as a preferred molecule of choice. See, de Fougerolles, A., Vornlocher, H.-P., Maraganore, J. & Lieberman, J. Interfering with disease: a progress report on siRNA-based therapeutics. Nat Rev Drug Discov 6, 443-453 (2007), which is incorporated by reference in its entirety. However, there are certain challenges for siRNA to be used as a therapeutic molecule, such as siRNA degradation in body fluids and the high molecular weight with anionic charge that does not allow for siRNA cellular uptake and penetration to the site of action: the cytoplasm. See, Whitehead, K. A., Langer, R. & Anderson, D. G. Knocking down barriers: advances in siRNA delivery. Nat Rev Drug Discov 8, 129-138 (2009), which is incorporated by reference in its entirety. These challenges can be overcome by employing delivery systems or carriers, and one way of doing it is by using counter-ion polyelectrolyte (e.g., PEI, chitosan, etc.) that can condense the siRNA into compact size, protecting the activity of siRNA, screen the negative charge to enhance the cellular accumulation, and ensure the endosomal escape of the siRNA for effective RNA interference. See, Richards Grayson, A., Doody, A. & Putnam, D. Biophysical and Structural Characterization of Polyethylenimine-Mediated siRNA Delivery in vitro. Pharm Res 23, 1868-1876 (2006), Swami, A. et al. A unique and highly efficient non-viral DNA/siRNA delivery system based on PEI-bisepoxide nanoparticles. Biochemical and Biophysical Research Communications 362, 835-841 (2007), and Malek, A. et al. In vivo pharmacokinetics, tissue distribution and underlying mechanisms of various PEI(-PEG)/siRNA complexes. Toxicology and Applied Pharmacology 236, 97-108 (2009), each of which is incorporated by reference in its entirety. Similar to any other polyelectrolyte interactions, the key issues are polydipersity, controllability, and batch-to-batch variability of nanoparticle formulations, which are more prominent on scale-up of siRNA/polycation polyplex nanoparticles. By using the coaxial turbulent jet mixer, synthesize the siRNA/PEI polyplex nanoparticles can be synthesized that are smaller and more uniform in size compared to the conventional bulk synthesis method (FIG. 13A). The siRNA/PEI polyplexes thus formed were lyophilized and stored at −20° C. until use. The transfection ability and resulting gene knockdown of the siRNA/PEI polyplex nanoparticles (molar ratio 1:3), thus formed was tested in dual-luciferase (firefly and renilla) expressing HeLa cell line and were found to reduce the luciferase expression to 50% and 40% at effective siRNA concentration of 20 μmol and 50 μmol, respectively (FIG. 13B). In contrast, the commercially available transfection agent Lipofectamine 2000 was able to reduce the luciferase expression to only 95% at a siRNA concentration of 20 μmol, which was brought down to 40% at 50 μmol siRNA concentration (FIG. 13B). This result illustrates the utility of the coaxial turbulent jet mixer to ensure batch-to-batch reproducibility of siRNA/PEI polyplex NPs while maintaining the effectiveness for gene knockdown.

Similar to other polyelectrolyte interactions, siRNA/polycation polyplex NP formulations prepared by bulk synthesis method have limited uniformity, batch-to-batch reproducibility, and scalability. In case of the conventional bulk synthesis method, scale-up for the synthesis of siRNA/polycation polyplex NPs is a conundrum because the physicochemical properties of NPs are significantly altered as the batch size is increased for scale-up, as illustrated by the observed differences in the size distributions of polyplex NPs prepared in different batch sizes using the conventional bulk synthesis method (FIG. 13A). In contrast, the coaxial turbulent jet mixer can synthesize siRNA/PEI polyplex NPs in a continuous and high-throughput manner, resulting in smaller NPs with narrower size distributions compared to those prepared by bulk synthesis (FIG. 13A). The properties of NPs synthesized using the coaxial turbulent jet mixer are independent of the batch size (i.e., amount of NPs synthesized) because the conditions for NP formation are identical and independent of the batch size for all NPs produced.

Until now, the application of nanoparticles, which is synthesized by microfluidic platform, to large animal in vivo and clinical studies has been challenging mainly due to the intrinsic problem of low production rates. Using the coaxial turbulent jet mixer, the production rate issue can be resolved without impairing controllability and reproducibility of microfluidic platform. When the coaxial turbulent jet mixer was operated at high Re and high concentration of PLGA-PEG precursor (i.e., 50 mg/mL) the production rate up to 2.19 g/min was achieved (Table 1), which is equivalent to 3.15 kg/d and 1.15 ton/yr. Considering that the production rates typically required for drug delivery applications in clinical studies and industrial scale productions of nanoparticles are order of 0.1 kg/d and 1 kg/d, respectively, it is noteworthy that a single coaxial turbulent jet mixer can meet the both requirements.

In addition, more homogeneous and smaller nanoparticles can be prepared by coaxial turbulent jet mixer compared to those synthesized by conventional bulk synthesis method, because τₘᵢₓ is more controllable and shorter than the τ_{agg}. Since smaller nanoparticles can penetrate more deeply into solid tumors (see, Wong, C. et al. Multistage nanoparticle delivery system for deep penetration into tumor tissue. Proc. Natl. Acad. Sci. U.S.A. 108, 2426-2431 (2011), Cabral, H. et al. Accumulation of sub-100 nm polymeric micelles in poorly permeable tumours depends on size. Nat. Nanotechnol. 6, 815-823 (2011), and Chauhan, V. P. et al. Normalization of tumour blood vessels improves the delivery of nanomedicines in a size-dependent manner. Nat. Nanotechnol. 7, 383-388 (2012), each of which is incorporated by reference in its entirety), the coaxial turbulent jet mixer has the potential to synthesize nanoparticles with better drug delivery performance compared to larger nanoparticles. As a proof of concept, siRNA/PEI polyplex nanoparticles were formulated using the coaxial mixer device that can knockdown the target gene more effectively as compared to the commercially available transfection agents, with a lower siRNA dose (FIG. 13B).

The coaxial turbulent jet mixer is simple and versatile platform for synthesis of various types of nanoparticles. As a proof of concept, the synthesis of PLGA-PEG nanoparticles, lipid vesicles, iron oxide nanoparticles, polystyrene nanoparticles, and siRNA/PEI polyplex nanoparticles that are widely used for biomedical applications were demonstrated. In addition, various functional agents including anti-cancer drug, insulin, fluorescent dye, and siRNA could be encapsulated while the nanoparticles were prepared. This mixer design can be used to synthesize the other type of nanoparticles and microparticles.

In summary, the coaxial turbulent jet mixer can be used as a versatile platform for reproducible and controlled synthesis of nanoparticles with high-throughput manner, which is required for the large animal in vivo studies, clinical trials, and industrial scale productions. The technology can expedite the personalized nanomedicines to the clinic and industrial scale production of nanoparticles.

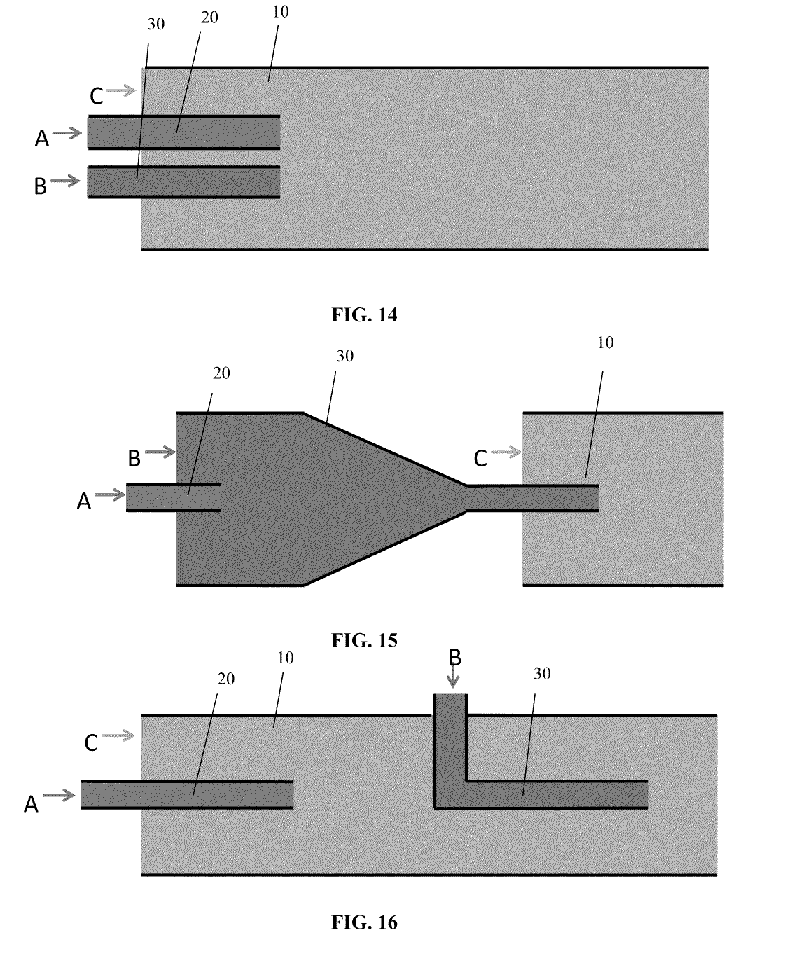

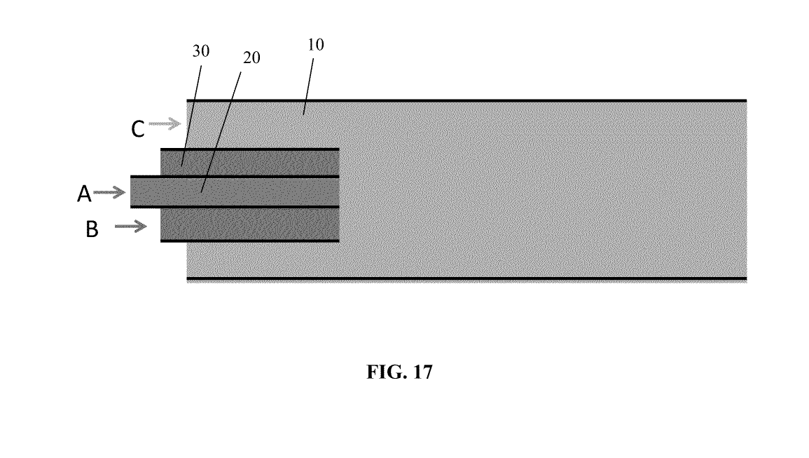

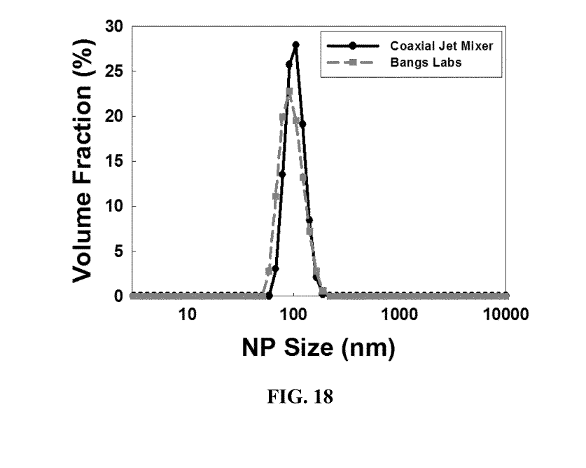

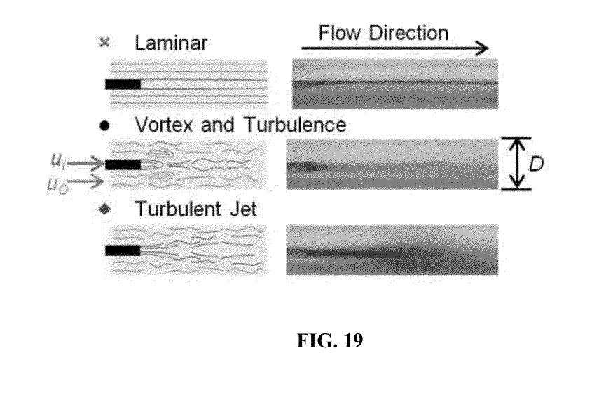

Multiple flows can be combined to create more complex nanoparticles or mixtures. The multiple flows can have three or more flows. The multiple flows can be introduced in parallel, or in series or sequentially. Different configurations can produce nanoparticle compositions having core-shell nanoparticle designs, or mixed compositions. Referring to FIG. 14, a mixer can include conduit 10 having flow C, into which two flows A and B are introduced simultaneously. Flow A can be a coaxial flow with flow C as is flow B. Nozzle 20 introduces flow A into conduit 10. Nozzle 30 can introduce flow B into flow C in conduit 10 adjacent to flow A. Nozzles 20 and 30 can enter conduit 10 from the same direction. Referring to FIG. 15, a mixer can include conduit into which flow A is coaxially introduced into flow B and this combined flow can be coaxially introduced into flow C. Nozzle 20 introduces flow A into nozzle 30 which can then be introduced into flow A in conduit 10. The flows can be mutually coaxial. Referring to FIG. 16, a mixer can include conduit 10 having flow C, into which two flows A and B are introduced. Flow A can be a coaxial flow with flow C. Nozzle 20 introduces flow A into conduit 10. Downstream of flow A, nozzle 30 can introduce flow B into flow A in conduit 10. Nozzle 30 can enter conduit 10 from the side and turn to provide coaxial flow. Referring to FIG. 17, a mixer can include conduit 10 having flow C, into which a coaxial flow with flow B is introduced. A mixer can also include flow A which is introduced to a conduit having flow B. Flow A can be a coaxial flow with flow C as is flow B. Nozzle 20 introduces flow A into conduit 10. Nozzle 30 can introduce flow B into flow C in conduit 10. Nozzles 20 and 30 can enter conduit 10 from the same direction.

In these examples, flow A can include nanoparticle precursors. Flow B can include a different nanoparticle precursor, a quenching solution, a nonsolvent or a surfactant. Each component can influence nanoparticle growth. In some embodiments, flow C includes a nonsolvent for nanoprecipitation. Additional flow inputs can be added downstream to create more complex mixtures.

Other embodiments are within the scope of the following claims.

What is claimed is:

1. A method for preparing nanoparticles comprising:
flowing a first stream of a first solution into a conduit, wherein the first solution contains precursors of the nanoparticles;
flowing a second stream of a second solution into the conduit; and
mixing the first stream and the second stream to form a mixed stream having a Reynolds number of between 300 and 1,000,000 in which the nanoparticles are formed.

2. The method of claim 1, wherein the formation of the nanoparticles is continuous.

3. The method of claim 1, wherein the nanoparticles are formed by nanoprecipitation or emulsion formation.

4. The method of claim 1, wherein a component of the first solution reacts with a component of the second solution.

5. The method of claim 1, wherein the nanoparticles are substantially uniformly distributed in the mixed stream after formation.

6. The method of claim 1, wherein the first stream is introduced within the second stream.

7. The method of claim 1, wherein the cross sectional area of the first stream is more than 1% of the cross sectional area of the conduit.

8. The method of claim 1, wherein the cross sectional area of the first stream is less than 90% of the cross sectional area of the conduit.

9. The method of claim 1, wherein the size of the nanoparticles is between 1 nm and 500 nm.

10. The method of claim 1, wherein the size of the nanoparticles is changed by changing the flow parameters of the first stream and the second stream.

11. The method of claim 1, wherein the mixed stream includes a vortex regime, a turbulence regime, or a turbulent jet regime.

12. The method of claim 1, wherein the flow behavior of the mixed stream includes turbulent jet flow.

13. The method of claim 1, wherein the flow velocity of the mixed stream varies.

14. The method of claim 1, wherein the Reynolds number of the mixed stream varies.

15. The method of claim 1, wherein a mixing timescale of the mixed stream is between 0.1 and 100 milliseconds.

16. The method of claim 1, wherein a flow velocity ratio of the first stream to the second stream is between 0.01 and 100.

17. The method of claim 1, wherein a volume ratio between the first solution and the second solution is between 10:1 and 1:100.

18. The method of claim 1, wherein the volume ratio between the first solution and the second solution is between 1:3 to 1:20.

19. The method of claim 1, wherein the nanoparticles include PLGA-PEG.

20. The method of claim 1, wherein the nanoparticles include iron oxide.

21. The method of claim 1, wherein the nanoparticles include polystyrene.

22. The method of claim 1, wherein the nanoparticles include siRNA/PEI polyplex.

23. The method of claim 1, wherein the nanoparticles include lipid vesicles.

24. The method of claim 1, wherein the nanoparticles contain a drug molecule.

25. The method of claim 1, wherein the nanoparticles contain a fluorescent molecule.

26. The method of claim 1, wherein the conduit is a tube.

27. The method of claim 1, wherein the second stream flows simultaneously with the first stream.

28. A device for preparing nanoparticles, comprising a conduit configured to introduce a first stream of a first solution into the conduit, a second stream of a second solution into the conduit at a mixing zone of the conduit, wherein the Reynolds number at the mixing zone is between 300 and 1,000,000.

29. The device of claim 28, wherein the device is a coaxial turbulent jet mixer.

30. A device for preparing nanoparticles, comprising a first conduit configured to introduce a first stream of a first solution into the first conduit, and a second conduit configured to introduce a second stream of a second solution into the second conduit, wherein the first conduit is inserted into the second conduit, and wherein the Reynolds number at the location of the introduction of the first solution is between 300 and 1,000,000.

31. The device of claim 30, wherein the device comprises a third conduit configured to introduce a third stream of a third solution, wherein the third conduit is inserted into the second conduit, and wherein the Reynolds number at the location of the introduction of the third solution is between 300 and 1,000,000.

32. The device of claim 31, wherein the location of the introduction of the first solution, the location of the introduction of the second solution, and the location of the introduction of third solution are controlled to control the time delay between the introduction of the first solution, the introduction of the second solution, and the introduction of the third solution.

33. The device of claim 30, wherein the device comprises a plurality of devices, each device comprising a first conduit configured to introduce a first stream of a first solution into the first conduit, and a second conduit configured to introduce a second stream of a second solution into the second conduit, wherein the first conduit is inserted into the second conduit, and wherein the Reynolds number at the location of the introduction of the first solution is between 300 and 1,000,000.

34. A method for preparing nanoparticles comprising:
introducing a first stream of a first solution into a first conduit, wherein the Reynolds number at the location of the introduction of the first solution is between 300 and 1,000,000;
introducing a second stream of a second solution into a second conduit, wherein the first conduit is inserted into the second conduit;
introducing a third stream of a third solution into a third conduit, wherein the third conduit is inserted into the second conduit, and wherein the Reynolds number at the location of the introduction of the third solution is between 300 and 1,000,000;
wherein the first solution or the second solution contains nanoparticle precursors, and wherein nanoparticles form when the first solution mixes with the second solution and the third solution.

35. A method for preparing nanoparticles comprising:
continuously flowing a first stream of a first solution into a conduit, wherein the first solution contains precursors of the nanoparticles; and
continuously flowing a second stream of a second solution into the conduit such that the second stream forms a turbulent jet within the first stream;
wherein the first stream and the second stream form a mixed stream having a Reynolds number of between 300 and 1,000,000 in which the nanoparticles are formed.

36. The claim of method of claim 1, wherein the second solution contains precursors of the nanoparticles.
